(19) United States
(12) Patent Application Publication
(10) Pub. No.: US 2003/0223957 A1
(43) Pub. Date: Dec. 4, 2003

(54) Biodegradable PEG based polymer formulations in ocular applications

(76) Inventors: Daniel M. Schwartz, San Francisco, CA (US); Keith G. Duncan, San Francisco, CA (US); Jay M. Stewart, Fort Lauderdale, FL (US)

Correspondence Address: FULBRIGHT & JAWORSKI, LLP, 1301 MCKINNEY, SUITE 5100, HOUSTON, TX, 77010-3095, US

(21) Appl. No.: 10/410,860

(22) Filed: Apr. 10, 2003

Related U.S. Application Data
(60) Provisional application No. 60/371,531, filed on Apr. 10, 2002.

(51) Int. Cl. A61K 31/765

(52) U.S. Cl. 424/78.38

(57) ABSTRACT

The present invention relates to methods and pharmaceutical compositions involving the use of bioerodible (biodegradable) polymers to address fundamental needs in ocular surgery including sealants and sealing methods, barriers to cellular adhesion and proliferation, and mechanical barriers. In a particular embodiment, the present invention is also directed to the treatment of intraocular hypotony in an eye by limiting the flow of aqueous from the eye. In a preferred embodiment, application of a polymer, such as to the angle of the eye, limits the flow, thereby increasing the intraocular pressure.

### CROSS-REFERENCE TO RELATED APPLICATIONS

This application claims the benefit of the filing date of U.S. Patent Provisional Application Serial No. 60/371,531, filed Apr. 10, 2002, which is incorporated herein by reference in its entirety.

### STATEMENT REGARDING FEDERALLY SPONSORED RESEARCH OR DEVELOPMENT

The present invention was developed at least in part with federal funds from NIH Grant No. 2 R44 EY12332-02. The U.S. Government may have certain rights in the invention.

### FIELD OF THE INVENTION

The present invention generally regards the field of medicine. More particularly, it regards the field of ophthalmology.

### BACKGROUND OF THE INVENTION

In the ocular surgery and eye care area, certain problems arise due to leakage, cellular adhesion and proliferation, and mechanical barrier issues. For example, in filtration surgery, a protected opening in the eye is created to normalize intraocular pressure, and the opening is often covered by a sutured conjunctival incision. If the closure of conjunctiva is not water tight, leakage can occur, which results in a pressure that is too low (hypotony) and which increases the risk of infection, the risk of choroidal hemorrhage, and/or of having a flat anterior chamber. In corneal surgery, leakage of aqueous humor occurs across an inadequately sutured wound. This may occur following trauma, corneal transplantation, or perforating infectious and non-infectious processes.

Examples of ocular procedures in which deleterious cellular adhesion and proliferation, among additional issues, arise include at least vitreoretinal surgery, filtration surgery, corneal transplantation surgery, conjunctival cicatricial disease, strabismus or scleral buckling surgery, and cataract surgery. In filtration surgery, one of the leading causes of failure is scarring of the filtration site and bleb caused by cellular and protein adhesion formation. Endothelial graft rejection is associated with binding of white blood cells and proteins to the endothelial surface of the cornea in corneal transplantation surgery. In conjunctival cicatricial disease, such as Stevens Johnson Syndrome, adhesions (symblepharon) form between the palpebral and bulbar conjunctival surfaces. In strabismus or scleral buckling surgery, adhesions commonly form between the extraocular muscles and adjacent surfaces (e.g. tenon's capsule, scleral, scleral buckle), which impair ocular motility and may cause double vision (diplopia). After cataract surgery, lens epithelial cells frequently migrate over the posterior lens capsule and cause posterior capsule opacification. Finally, in dry eye the ocular surface is not adequately wetted by an optimized tear film, which produces ocular irritation and increases the chance of corneal infection. Thus, patients suffering from these conditions are in need of a effective method for addressing problems that arise due to these leakage, cellular adhesion and proliferation, and/or mechanical barrier issues.

U.S. Pat. No. 5,587,175 regards gels as a vehicle for drug delivery, particularly for protective corneal shields or as ablatable corneal masks useful in laser reprofiling of the cornea. The compositions are also useful in the absence of drug delivery such as for separating surgically or injured tissue as a means of preventing adhesions.

U.S. Pat. Nos. 4,938,763 (and the subsequent reexamination) and 5,739,176 are directed to methods and compositions for a biodegradable, in situ-forming implant, particularly for use as a controlled-release delivery system for a biological agent. More specifically, in some embodiments a thermoplastic polymer, such as a copolymer of polyethylene glycol with polylactide, polyglycolide, polycaprolactone, or a terpolymer thereof, is dissolved in a water-soluble organic solvent, and the composition is then placed into an implant site, wherein the organic solvent dissipates or diffuses into body fluid, and the thermoplastic polymer coagulates to produce the solid implant.

U.S. Pat. No. 6,149,931 regards methods, compositions and articles of manufacture for the closure of retinal breaks. More particularly, the compositions and methods are directed to a non-toxic polymer formulation comprising a polymer precursor and its transformation into a gel-like coat, such as by photochemical reaction.

Another specific aspect of the present invention relates to the treatment of ocular hypotony. Hypotony, defined as intraocular pressure less than 5 mmHg, is a common problem in ophthalmology. In the eye, the normal rate of aqueous humor production is 2.5 μL/min, and about 90% of aqueous exits through the trabecular meshwork/Schlemm canal, while about 10% exits through uveoscleral outflow. When intraocular pressure declines below the episcleral venous pressure, usually about 9 mm Hg, flow through the conventional route ceases. Thus, uveoscleral outflow predominates at low intraocular pressures.

In general, hypotony occurs when aqueous production is not balanced with outflow. Outflow may be greater than usual in conditions such as wound leak, overfiltering bleb, or cyclodialysis cleft. Conditions that alter ciliary body function, such as iridocyclitis or hypoperfusion, may cause insufficient aqueous production.

Inflammation plays a key role in the evolution of hypotony. It causes increased permeability of the blood-aqueous barrier. Choroidal fluid is believed to accumulate as a result of enhanced uveoscleral outflow and decreased aqueous humor production, a cycle that often is perpetuated once choroidal effusions develop.

The clinical history of patients with hypotony frequently includes recent trauma or surgery, especially primary glaucoma surgery with antimetabolites; or a history of iridocyclitis, blurred vision, or eye pain (usually a deep ache that is present particularly if choroidal detachment has occurred). Physical clinical characteristics associated with hypotony include some or all of the following: Seidel positive wound leak; large bleb following trabeculectomy or tube shunt; inadvertent postoperative filtering bleb; inflammatory cells and flare in the anterior chamber; shallowing of the anterior chamber (corneal decompensation, synechiae formation, corneal astigmatism); accelerated cataract formation; ciliochoroidal detachment (serous or hemorrhagic); cyclodialysis cleft; hypotony maculopathy (retinal folds, vascular engorgement and tortuosity, or optic disc swelling); and retinal detachment.

There are a variety of causes of unilateral hypotony. In exemplary embodiments, these include wound leak; overfiltering or inadvertent bleb; cyclodialysis cleft; inflammation (iridocyclitis or trauma); retinal detachment; ocular ischemia; scleral perforation with neddle or suture; scleral rupture following trauma; chemical cyclodestruction from antimetabolites photocoagulation or cryoablation of the ciliary body; pharmacologic aqueous suppression; bilateral hypotony; systemic hypertonicity or acidosis (dehydration, uremia, uncontrolled diabetes, or use of hyperosmotic agents); or myotonic dystrophy.

In diagnosis of ocular hypotony, imaging studies are useful, including ultrasonic biomicroscopy which can evaluate further anterior chamber depth, position of the ciliary body, and presence of anterior ciliary detachment. B-mode ultrasonography is also useful when the fundus is not visualized easily and can assist in determining the size and extent of ciliochoroidal detachment, choroidal hemorrhage, and retinal detachment. Additional tests include those wound leaks, such as are identified by Seidel testing.

Some cases of hypotony are treatable. For example, when low intraocular pressure results from excessive egress of aqueous fluid from the eye due to a wound leak or excessive filtration through a trabeculectomy bleb, surgical intervention often restores normal pressure. Insufficient production of aqueous fluid can also cause hypotony. When this is due to intraocular inflammation, anti-inflammatory medications may lead to normalization of pressure as the underlying process is treated. If a cyclodialysis cleft follows trauma or intraocular surgery, ciliary body dysfunction leading to hypotony can often be managed by repair of the cleft surgically or with laser or cryopexy (Kuchle and Naumann, 1995).

Topical anti-inflammatory agents, especially prednisolone acetate 1%, may be useful in many types of hypotony. Non-steroidal anti-inflammatory agents may be used adjunctively. Cycloplegic agents often are indicated in swollen eyes. Topical broad-spectrum antibiotics are appropriate with wound leaks and in recent surgery or trauma cases. Particularly, drugs including corticosteroids (anti-inflammatory agents), mydriatic/cycloplegics (which relax any ciliary muscle spasm that can cause a deep aching pain and photophobia), or non-steroidal anti-inflammatory agents (having both analgesic and anti-inflammatory actions) are useful for hypotony.

Although some cases are fairly treatable, many cases of hypotony are more difficult to treat. Ciliary body dysfunction and hyposecretion of aqueous can occur after vitreoretinal surgery for retinal detachment, especially in cases complicated by proliferative vitreoretinopathy (Lewis et al., 1991; Lewis and Aaberg, 1991). Hypotony has been found to be the most common cause of functional failure in these cases once the retina has been successfully reattached (Zarbin et al., 1991). In this setting, traction on the ciliary body by the anterior vitreous base due to abnormal proliferative tissue is thought to damage the ciliary epithelium directly and cause low-lying detachment of the ciliary body (Lewis et al., 1991; Lewis and Aaberg, 1991; Zarbin et al., 1991; Lewis and Verdaguer, 1996). Large retinotomies have also been proposed to contribute to hypotony via increased clearance of fluid from the eye by the pumping action of the retinal pigment epithelium (Kirchhof, 1994). Although some pressure increase through anatomic reattachment of the ciliary body has been reported after meticulous dissection of epiciliary proliferative tissue (Zarbin et al., 1991; Lewis and Verdaguer, 1996), most cases of hypotony after retinal detachment surgery are refractory to current treatment methods. Topical steroid drops are usually used, with limited effect.

The visual consequences of untreated hypotony can be dire. Folds in the anterior and posterior segment can develop, and serous retinal detachment can occur. The anterior segment can shrink, and the eye can progress to a stable pre-phthisis state or to frank phthisis bulbi. In these cases, the eye not only provides no useful vision but is also cosmetically unappealing and can be painful (Lewis and Aaberg, 1991).

U.S. Pat. No. 5,700,794 describes a method for treating ocular hypotony by administering topically to the eye a pharmaceutically effective amount of a mineralocorticoid, such as aldosterone, dihydrocortisol, fludrocortisone, or 11-desoxycorticosterone, preferably at a concentration of 0.05-5 wt. %.

U.S. Pat. No. 6,274,614 is directed to a method for reducing or preventing the effects of inflammation arising from injury to eye tissue following glaucoma surgery, wherein the eye tissue is contacted with a photosensitizing agent capable of penetrating into the injured tissue, followed by exposing the contacted tissue to light having a wavelength absorbed by the photosensitizing agent for a time sufficient to reduce or prevent inflammation in the exposed tissue.

U.S. Pat. No. 4,328,803 is directed to protecting eye structures following surgery by introducing into the anterior segment of the eye a given volume of a solution containing a sufficient concentration of sodium hyaluronate to protect eye tissue, wherein diluting the volume in the site thereby reduces the concentration thereof prior to closure such that abnormally high post-operative intra-ocular pressure within the human eye is avoided.

U.S. Pat. No. 5,360,399 regards a method of maintaining a constant pressure in the eye associated with the aqueous humour by making a lamellar incision of the sclera for exposing a section of Schlemm's canal and injecting the highly viscous sodium hyaluronate into the canal by means of a tube introduced into the canal for opening the trabecular tissue traumatically by a hydraulic expansion at one or more points.

U.S. Pat. No. 6,636,585 is directed to a method for conducting ocular surgery, comprising introducing an aqueous solution of sodium hylauronate into an eye as a surgical aid.

Anterior chamber injections of sodium hyaluronate have been reported in patients with hypotony due to choroidal detachments and uveitis (Cadera and Willis, 1982; Daniele and Schepens, 1989; Koerner et al., 1985). In these cases, increases in intraocular pressure occurred following the procedure, suggesting that the physical obstruction of aqueous outflow by sodium hyaluronate is useful for restoring normal intraocular pressure for patients with hypotony. However, sodium hyaluronate remains in the eye for only a short time; the half-life is 75 minutes regardless of the molecular weight, although some increase is obtained by varying the viscosity (Laurent and Fraser, 1983; Schubert et al., 1984).

Thus, there exists a need in the art for procuring and/or maintaining sufficient intraocular pressure for a sustainable period of time, preferably utilizing at least one biodegradable polymer.

### SUMMARY OF THE INVENTION

The present invention is directed to compositions, methods, and articles of manufacture relating to the use of bioerodible (biodegradable) PEG-based polymer formulations to address fundamental needs in ocular surgery. For example, the invention discloses the use of such polymers as 1) sealants; 2) barriers to cellular adhesion and proliferation; and/or 3) mechanical barriers. The invention provides methods of treatment in a mammal comprising applying to a subject location a non-toxic polymer formulation comprising at least one polymer precursor, and transforming the polymer formulation into a gel-like coat. In a preferred embodiment, the polymer formulation comprises a photochemically reactive polymer precursor species that can be transformed from a liquid to gel form by exposure to light. The polymer may also be transformed into a gel form by applying another type of stimulus, such as a chemical. The polymer may be autopolymerizable, in some embodiments. Another preferred composition includes a mixture of two mutually reactive polymer precursors.

This invention provides new uses for the compounds and compositions disclosed (e.g., bioerodible (biodegradable) polymers) in U.S. Pat. No. 6,149,931 (Schwartz et al., Methods and Pharmaceutical Compositions for the Closure of Retinal Breaks, issued November 21, 2000) which is herein incorporate by reference in its entirety.

The instant invention provides, for example, methods for sealing openings from filtration surgery; sealing sutures from corneal surgery to prevent leakage of aqueous humor; sealing openings made in vitreoretinal surgery; preventing cellular and protein adhesion, for example, preventing scarring of the filtration site and bleb following filtration surgery; preventing corneal graft rejection associated with corneal transplants; preventing adhesions in conjunctival cicatricial disease; and preventing adhesions after strabismus or scleral bucking surgery and after cataract surgery; and forming mechanical barriers, for example, for treating dry eye, wherein the methods utilize bioerodible (biodegradable) polymers.

It is contemplated that embodiments discussed herein with respect to one method of the invention may be implemented with respect to other methods of the invention.

As indicated above, the present invention in particular embodiments is useful for treating ocular hypotony, and in some embodiments this utilizes a polymer as described herein. As described above, hypotony results from an imbalance between the secretion and drainage of aqueous fluid in the eye. In most cases of hypotony, subnormal amounts of aqueous are produced in an eye that has a normal trabecular meshwork, the principal outflow site of aqueous in the eye. As described above, efforts to treat hypotony have generally been directed toward increasing the production of aqueous fluid. The present invention regards a technique in which the intraocular pressure in hypotonous eyes is increased by limiting the outflow of aqueous from the eye.

Although other methods address restoration of intraocular pressure in the eye, the methods of the present invention are directed to long-term solutions for obtaining a similar goal. Specifically, polymers, such as photopolymerizable polymers, are utilized to allow for sustained increases in intraocular pressure, since the biodegradable polymers can be formulated to last for months or longer before degradation occurs.

In one embodiment of the present invention, there is a method for providing a polymer to an ocular defect in a mammal, wherein the ocular defect is other than a retinal break, comprising applying over and/or around the ocular defect a non-toxic polymer formulation comprising at least one polymer precursor that is a poly(ethylene glycol) (PEG) based polymer precursor; and transforming the polymer formulation into a gel-like coat. In a specific embodiment, the polymer provides a water tight seal to the ocular defect, which may be at least one opening, incision, wound, hole, tear, gap, notch, aperture, cavity, cut, slit, scratch, injury, lesion, gash, abrasion, break, puncture, perforation, rip, or split in at least one eye tissue. The ocular defect may be an indirect or direct result of a disease, medical condition, or surgery. In some embodiments, the disease is Stevens Johnson Syndrome, or ocular pemphigoid, such as following alkalai burn to ocular surface. In some embodiments, the surgery is filtration surgery, vitreoretinal surgery, corneal surgery, scleral buckling surgery, cataract surgery. In some embodiments, the medical condition is dry eye.

In an additional embodiment of the present invention, there is a method of sealing an opening in an eye of a mammal, wherein the opening is not a retinal break, comprising applying over and/or around an opening in the eye a non-toxic polymer formulation comprising at least one polymer precursor that is a poly(ethylene glycol) (PEG) based polymer precursor; and transforming the polymer formulation into a gel-like coat, wherein the coats forms a seal. In a specific embodiment, the seal is water tight or reduces the flow of a liquid from the opening, and/or is resistant to intraocular pressure from the eye. The opening may be optionally sutured, it may be associated with filtration surgery, it may be associated with corneal surgery, and/or it may be associated with a postoperative glaucoma filtration bleb or conjunctival buttonhole, in some embodiments.

In another embodiment of the present invention, there is a method for sealing an opening in an eye of a mammal caused by filtration surgery, comprising applying over and/or around an opening in the eye a non-toxic polymer formulation comprising at least one polymer precursor that is a poly(ethylene glycol) (PEG) based polymer precursor; and transforming the polymer formulation into a gel-like coat, wherein the coat forms a seal. The seal may reduce flow from the opening and/or resistant to intraocular pressure from the eye. The opening may be optionally sutured and/or may be a conjunctival incision.

In an additional embodiment of the present invention, there is a method of sealing a conjunctival incision in the eye of a mammal following filtration surgery, comprising applying over and/or around the conjunctival incision a non-toxic polymer formulation comprising at least one polymer precursor that is a poly(ethylene glycol) (PEG) based polymer precursor; and transforming the polymer formulation into a gel-like coat, wherein the coats forms a seal. The seal may be water tight and/or resistant to intraocular pressure from the eye. In a specific embodiment, the incision is optionally sutured. In another specific embodiment, the seal biodegrades after about 2-16 weeks.

In an additional embodiment of the present invention, there is a method of sealing a leaking corneal wound in the eye of a mammal, comprising applying to the wound a non-toxic polymer formulation comprising at least one polymer precursor that is a poly(ethylene glycol) (PEG) based polymer precursor; and transforming the polymer formulation into a gel-like coat, wherein the coat forms a seal. In a specific embodiment, the seal reduce flow from the wound and/or resistant to intraocular pressure from the eye. The wound may be optionally sutured, and the seal biodegrades after about 2-16 weeks, in some embodiments.

In an additional embodiment of the present invention, there is a method of sealing sclerotomies from vitreoretinal surgery in the eye of a mammal, comprising applying to a sclerotomic wound a non-toxic polymer formulation comprising at least one polymer precursor that is a poly(ethylene glycol) (PEG) based polymer precursor; and transforming the polymer formulation into a gel-like coat, wherein the coat forms a seal. The seal may reduce flow from the wound and/or resistant to intraocular pressure from the eye. The wound may be optionally sutured and/or the seal may biodegrade after about 2-16 weeks.

In another embodiment of the present invention, there is a method of forming at least one barrier to adhesion and/or of preventing cellular adhesion and proliferation in the eye of a mammal, comprising applying to a surface in the eye a non-toxic polymer formulation comprising at least one polymer precursor that is a poly(ethylene glycol)(PEG) based polymer precursor; and transforming the polymer formulation into a gel-like coat. In a specific embodiment, the surface is a filtration site and bleb following filtration surgery. In an additional specific embodiment, the prevention of adhesion reduces scarring of the filtration site and bleb.

In another embodiment of the present invention, there is a method of preventing adhesions from forming between two apposing tissue surfaces in the eye of a mammal, comprising applying to apposing surfaces in the eye a non-toxic polymer formulation comprising at least one polymer precursor that is a poly(ethylene glycol)(PEG) based polymer precursor; and transforming the polymer formulation into a gel-like coat. In a specific embodiment, the surface is the filtration site and bleb following filtration surgery. In a further specific embodiment, the prevention of adhesion reduces scarring of the filtration site and bleb. The coat may biodegrade after about 2-16 weeks, in some embodiments. In a specific embodiment, both the outside and inside surfaces of a scleral flap as well as the scleral margins surrounding an excised trabecular segment are coated with the non-toxic polymer formulation.

In another embodiment of the present invention, there is a method for reducing scarring of the filtration site and bleb following filtration surgery in the eye of a mammal, comprising preventing and/or reducing post-operative adhesions by applying to apposing surfaces following surgery a non-toxic polymer formulation comprising at least one polymer precursor that is a poly(ethylene glycol)(PEG) based polymer precursor; and transforming the polymer formulation into a gel-like coat. The coat may biodegrade after about 2-16 weeks, in some embodiments. In a specific embodiment, both outside and inside surfaces of a scleral flap as well as the scleral margins surrounding an excised trabecular segment are coated with the non-toxic polymer formulation.

In an additional embodiment of the present invention, there is a method for preventing adhesions (symblepharons) from forming between the palpebral and bulbar conjunctival surfaces in conjunctival cicatricial disease such as Stevens Johnson Syndrome, comprising applying to the surfaces a non-toxic polymer formulation comprising at least one polymer precursor that is a poly(ethylene glycol)(PEG) based polymer precursor; and transforming the polymer formulation into a gel-like coat. In a specific embodiment, the coat biodegrades after about 2-16 weeks.

In another embodiment of the present invention, there is a method for preventing adhesions between the extraocular muscles and adjacent surfaces in strabismus or scleral buckling surgery, comprising applying to exposed extraocular muscles and adjacent tissue or prosthetic surfaces a non-toxic polymer formulation comprising at least one polymer precursor that is a poly(ethylene glycol) (PEG) based polymer precursor; and transforming the polymer formulation into a gel-like coat. In a specific embodiment, the adjacent surfaces are tenon's capsule, sclera, scleral buckle, or a combination thereof. In another specific embodiment, the coat biodegrades after about 4-6 weeks.

In an additional embodiment of the present invention, there is a method for preventing adhesions following corneal transplant surgery, comprising applying to the endothelial surface of the donor cornea prior to suturing to the host during keratoplasty surgery a non-toxic polymer formulation comprising at least one polymer precursor that is a poly(ethylene glycol)(PEG) based polymer precursor; and transforming the polymer formulation into a gel-like coat. In a specific embodiment, the white blood cells are prevented from adhering to the endothelial surface. In a further specific embodiment, the rejection of the corneal transplant is reduced due to the reduced adherence.

In another embodiment of the present invention, there is a method of preventing, after cataract surgery, lens epithelial cells from migrating over the posterior lens capsule and causing posterior capsule opacification, comprising applying to the endothelial surface of the donor cornea prior to suturing to the posterior capsule prior to implantation of the intraocular lens a non-toxic polymer formulation comprising at least one polymer precursor that is a poly(ethylene glycol)(PEG) bsed polymer precursor; and transforming the polymer formulation into a gel-like coat. In a specific embodiment, the coat biodegrades after about 6-48 months.

In an additional embodiment of the present invention, there is a method of forming a biodegradable mechanical barrier in the eye of mammal, comprising applying to an ocular surface to be protected a non-toxic polymer formulation comprising at least one polymer precursor that is a poly(ethylene glycol)(PEG) based polymer precursor; and transforming the polymer formulation into a gel-like coat. In a specific embodiment, the coating of the ocular surface alleviates ocular symptoms of irritability and protects the integrity and normal function of the ocular surface. In another specific embodiment, the coating provides protection against infection. In a further specific embodiment, the coat alleviates symptoms of dry eyes. In another specific embodiment, the transforming is by photopolymerization of the polymer precursor.

In a specific embodiment, the polymer formulation comprises a polymer precursor of the formula Pₘ-DₙWₒ-Dₚ-P_{q}, wherein W is a water-soluble polymer; D is a degradable moiety; P is a photopolymerization moiety; m and q are integers from 1 to about 10; o is an integer from 1 to about 100; and n and p are integers from 0 to about 120. In a specific embodiment, the PEG comprises reactive termini, such as, for example, free radical polymerizable termini or acrylate termini. In another specific embodiment, the PEG comprises a long chain PEG having a molecular weight of at least about 8,000 g/mol or, alternatively, having a molecular weight of at least about 20,000 g/mol.

The PEG based polymer precursor may further comprise degradable regions, and the degradable regions may comprise from about 0.5% to about 20% oligolactic acid. In a specific embodiment, the PEG based polymer precursor further comprises about 1% oligolactic acid.

In a specific embodiment of the present invention, a method further comprises applying at least one photoinitiator to the surface, such as, for example, an eosin Y photoinitiator. In another specific embodiment, the formulation further comprises at least one co-catalyst. In a further specific embodiment, the formulation further comprises at least one photoinitiator and at least one co-catalyst. In an additional specific embodiment, the formulation further comprises at least one photoinitiator, N-vinlypyrrolidone and triethanolamine.

The transformation may be by auto-polymerization of the polymer precursor, in a specific embodiment. The polymer formulation may comprise a first polymer precursor and a second polymer precursor, the first and second polymer precursors being mutually reactive. In a specific embodiment, the first polymer precursor is an amine, such as, for example, a tetra-amino poly(ethylene gylcol)(PEG). In a specific embodiment, the first polymer precursor is a protein and the second polymer precursor is a terminally-functionalized poly(ethylene glycol)(PEG). The protein may be albumin, collagen, or gelatin. In a specific embodiment, the protein is albumin. The second PEG molecule is a di-N-hydroxysuccinimidyl PEG, in a specific embodiment. The second polymer precursor is a hydroxysuccinimidly activated succinate-terminated PEG or carbonate-terminated PEG, in other specific embodiments. In a further specific embodiment, the gel-like coat of the present invention comprises a biodegradable polymer.

In an additional embodiment of the present invention, there is a method for increasing intraocular pressure in an eye, comprising the step of limiting the loss of aqueous from the eye. In a specific embodiment, loss from the eye is limited to substantially zero. The limiting step may be further characterized as applying a biocompatible polymer to the eye. In a specific embodiment, the application of the polymer is to the angle of the eye, in the posterior chamber of the eye, or both. In a specific embodiment, the application of said polymer obstructs the trabecular meshwork of said eye.

The polymer may be a photopolymerizable polymer, an autopolymerizable polymer, a polyethylene glycol-based polymer, or a combination thereof. In a specific embodiment, the polyethylene glycol-based polymer comprises poly(ethylene glycol)-cotrimethylene carbonate-co-lactide (M, 20,000) with acrylated end groups.

In a specific embodiment of the present invention, the half-life of the polymer is at least about 3 days.

In a specific embodiment, the applying step is further defined as removing aqueous from the eye; applying an apparatus to facilitate directing a polymer precursor into the angle; administering the polymer precursor; and polymerizing said polymer. In a specific embodiment, the aqueous is removed from the anterior chamber of the eye. In another specific embodiment, the removing step comprises removing the aqueous fluid from the anterior chamber with a needle. In a further specific embodiment, applying an apparatus step is further defined as injecting an air bubble into the anterior chamber.

In an additional specific embodiment, the method is further defined as applying a paracentesis configuration. In a further specific embodiment, the applying an apparatus step is through said paracentesis configuration. The method may further comprise application of anesthesia, such as at least one applied topically.

In a specific embodiment, the polymerizing step is further defined as applying light to a photopolymerizable polymer. The eye is in a human, in specific embodiments. The method may be repeated following reduction in intraocular pressure in the eye and/or the method may be repeated following degradation of the polymer.

In an additional embodiment of the present invention, there is a method for increasing intraocular pressure in an eye of a mammal, comprising the steps of removing aqueous from the eye; injecting an air bubble into the angle of the eye; administering a precursor of poly(ethylene glycol)-cotrimethylene carbonate-co-lactide with acrylated end groups into the angle; and polymerizing the precursor.

In another embodiment of the present invention, there is a method of hindering the loss of aqueous from the eye of an individual comprising administering a biocompatible polymer into the eye.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 illustrates an exemplary schematic PEG-based photochemically reactive polymer.

FIG. 2 demonstrates rabbit intraocular pressure following methods of the present invention utilizing a PEG-based polymer vs. control.

### DETAILED DESCRIPTION OF THE INVENTION

1. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “angle” as used herein refers to the area in the anterior chamber of the eye wherein the cornea and iris join. The angle is comprised of several structures that are components of the eye's drainage system, including the outermost part of the iris, the front of the ciliary body, the trabecular meshwork, and the Canal of Schlemm. The anterior chamber angle extends 360 degrees at the perimeter of the iris.

The term “aqueous” as used herein refers to the clear fluid, comprising at least water, inside the eye (the anterior and posterior chambers, particularly between the lens and cornea). It is renewed approximately every 100 minutes.

The term “hypotony” as used herein refers to intraocular pressure less than about 5 mmHg.

The term “intraocular pressure” as used herein is defined as the pressure in the eye determined by the production and drainage of aqueous fluid. In specific embodiments, the normal range of intraocular pressure is from about 9 to about 21 mmHg.

The term “ocular defect”, as used herein, refers to at least one opening, incision, wound, hole, tear, gap, notch, aperture, cavity, cut, slit, scratch, injury, lesion, gash, abrasion, break, puncture, performation, rip, split, and so forth in at least one eye tissue. In a preferred embodiment, the ocular defect is in a mammal, such as a human.

The term “polyethylene oxide” as used herein refers to poly(ethylene glycol) of molecular weight greater than about 20,000 Daltons (Da).

The term “seal” as used herein refers to reducing liquid flow from an ocular defect or reducing flow through and/or around a polymer applied to an ocular defect. The liquid may be aqueous.

The term “water tight” as used herein refers to the prevention of liquid, such as aqueous (water and/or saline) based liquid, from passage through and/or around a tissue, opening, cleavage, wound site, suture, and so forth, particularly of an ocular defect, utilizing a PEG-based polymer. In particular embodiments, “water tight” refers to substantially no leakage of the liquid through and/or around the polymer. The term “substantially no leakage” refers to there being no leakage or having leakage at less than or equal to 0.1 milliliter/min. In another specific embodiment, there is a leakage rate that maintains normal, or at least, improved (higher) intraocular pressure.

2. The Present Invention

This invention pertains to the field of ophthalmology, particularly to the application of bioerodible PEG-based polymer formulations for a variety of ocular therapies. In specific embodiments, the polymer formulations are utilized as sealants, barriers to cellular adhesion and proliferation, and/or mechanical barriers.

### A. PEG-Based Polymer Formulations as Sealants

Applications for PEG-based formulations as sealants include, for example, filtration surgery, corneal surgery, vitreoretinal surgery, and so forth. For example, sealants are used in filtration surgery, where a protected opening in the eye is created to normalize intraocular pressure. The opening is covered by a sutured conjunctival incision. If the closure of conjunctiva is not water tight, leakage can occur. This results in a pressure that is too low (hypotony) and increases the risk of infection, the risk of choroidal hemorrhage, and/or of having a flat anterior chamber. PEG-based polymer formulations (such as, for example, photo-polymerizing or chemical polymerizing) are applied to the conjunctival incision either alone or in conjunction with a suture-based closure to prevent leakage. After about 2-16 weeks, the PEG-based sealant biodegrades. Similar applications to a glaucoma filtration bleb are performed in the post-operative period to seal a leaking bleb or a conjunctival buttonhole.

Sealants are also utilized in corneal surgery, where leakage of aqueous humor can occur across an inadequately sutured wound. This may occur following trauma, corneal transplantation, or perforating infectious and non-infectious processes. PEG-based polymer formulations (such as, for example, photo-polymerizing or chemical polymerizing) are applied to the leaking corneal wound either alone or in conjunction with a suture-based closure to prevent leakage. After about 2-12 weeks, the PEG-based sealant biodegrades. No leakage occurs because fibrous proliferating has sealed the wound.

Also, sealants are used in vitreoretinal surgery, wherein sclerotomies are generally closed with sutures. As an alternative, PEG-based polymer formulations (such as, for example, photo-polymerizing or chemical polymerizing) are applied to the scleral incision to achieve closure without the use of sutures. After about 2-16 weeks, the PEG-based sealants biodegrades.

### B. PEG-Based Polymers as Barriers to Cellular Adhesion and Proliferation

PEG-based polymer formulations may also be used as barriers to cellular adhesion and/or to cellular proliferation. For example, infiltration surgery, one of the leading causes of failure is scarring of the filtration site and bleb. To prevent or lessen formation of post-operative adhesions, PEG-based polymer formulations (such as, for example, photo-polymerizing or chemical polymerizing) are applied to the apposing tissue surfaces during surgery. Because the biodegradable polymers prevent cellular and protein adherence, adhesion formation is diminished compared to untreated patients. After about 2-16 weeks, the PEG-based sealant biodegrades. Because of the adhesion protection offered during the acute post-operative period, adhesion formation after polymer biodegradation is minimal.

PEG-based polymer formulations are also used as barriers to cellular adhesion and proliferation in corneal transplantation surgery, wherein endothelial graft rejection is associated with binding of white blood cells and proteins to the endothelial surface of the cornea. To prevent such rejections, PEG-based polymer formulations (such as, for example, photo-polymerizing or chemical polymerizing) are applied to the endothelial surface of the donor cornea prior to suturing to the host during keratoplasty surgery. Because the biodegradable polymers prevent cellular and protein adherence, cellular and protein adhesion is diminished compared to untreated patients. After about 2-24 weeks, the PEG-based sealant biodegrades. Because of the barrier to cellular and protein adhesion provided by the biodegradable polymers during the post-operative period, the likelihood of graft rejection is minimized.

Biodegradable polymers as barriers to cellular adhesion and proliferation are furthermore used in conjunctival cicatricial disease, such as Stevens Johnson Syndrome, where adhesions (symblepharon) form between the palpebral and bulbar conjunctival surfaces. To prevent such adhesion formation, PEG-based polymer formulations (such as, for example, photo-polymerizing or chemical polymerizing) are applied to the bulbar and palpebral conjunctival surfaces. These biodegradable formulations bioerode in about 1-24 weeks. Repeated applications are used if an inflammatory process persists. By preventing adhesions formation, the biodegradable polymer formulations minimize damage to the ocular surface.

In strabismus or scleral buckling surgery, biodegradable polymers are utilized as barriers for cellular adhesion and proliferation, wherein adhesions commonly form between the extraocular muscles and adjacent surfaces (e.g. tenon's capsule, scleral, scleral buckles). These adhesions impair ocular motility and may cause double vision (diplopia). To prevent such adhesion formation, PEG-based polymer formulations (such as, for example, photo-polymerizing or chemical polymerizing) are applied to exposed extraocular muscles and adjacent tissue or prosthetic surfaces. These biodegradable formulations bioerode in about 2-16 weeks, after the acute post-operative period has passed and stimuli to adhesion formation are mitigated.

Another example of biodegradable polymers as barriers to cellular adhesion and proliferation is after cataract surgery, wherein lens epithelial cells frequently migrate over the posterior lens capsule and cause posterior capsule opacification. To prevent such opacification, PEG-based polymer formulations (such as, for example, photo-polymerizing or chemical polymerizing) are applied to the posterior capsule during cataract surgery. These biodegradable formulations bioerode in about 6-48 months, after the biological stimuli prompting posterior capsule opacification subside.

### C. PEG-Based Polymers as Mechanical Barriers

Finally, biodegradable polymers are used as mechanical barriers in some embodiments of the present invention. For example, in dry eye the ocular surface is not adequately wetted by an optimized tear film. This produces ocular irritation and increases the chance of corneal infection. To prevent the discomfort associated with dry eye and the attendant risk of intraocular infection, PEG-based polymer formulations (such as, for example, photo-polymerizing or chemical polymerizing) are applied to the ocular surface, such as in a drop formulation. These biodegradable formulations bioerode in about 1-30 days and may be periodically reapplied.

### D. PEG-Based Polymers

The invention provides a superior alternative to known methods and compositions in the art. The polymer formulation is applied in liquid form, assuring conformity to irregular tissue surfaces. It is then transformed to a thin, gel-like coat by, for example, photopolymerization with a light source or by chemical polymerization. Alternatively, a liquid polymer precursor that auto-polymerizes is applied over the tissue in question. The polymerized gel is bound to the ocular tissue and resists displacement with overlying turbulent fluid flow. In some embodiments, it is water permeable and allows diffusion of small molecules such as oxygen, glucose and other essential nutrients.. The polymerized gel can be formulated with different pore sizes to allow more or less water to diffuse through. In some embodiments, the polymerized gel is referred to as not being water tight, as water can diffuse, very slowly, through the polymer. In some embodiments, while the polymer adheres to the tissue it provides a seal and/or diminishes fluid from passing through or around the seal. In another specific embodiment, the seal prevents diffusion of aqueous slow enough to enable a functional closure of a defect. The term “functional closure of a defect” as used herein refers to enabling normal function of at least one ocular tissue.

One aspect of the invention is a method for sealing (which may also be referred to as closing or fastening) an ocular defect, such as an opening, an incision, a wound, a hole, a tear, and so forth, comprising applying a non-toxic polymer formulation to the ocular surface in question of the animal over and around the defect, and transforming the polymer formulation into a gel-like coat. Preferably, the resultant gel-like coat comprises a biodegradable polymer. In a specific embodiment, the ocular defect is not a retinal break or retinal detachment. Preferably the animal is a laboratory animal or domesticated animal, is more preferably a mammal, and most preferably is a human. Suitable laboratory animals include mice, rats, rabbits, monkeys, apes and other research animals. Suitable domesticated animals include dogs, cats, cattle, horses, goats, sheep, pigs, mules, donkeys, and other animals in the service or company of man.

A key feature of the requirements for the materials to be used in ocular surgery is that they adhere to the ocular defect over and around the break. One way to provide for this feature is to produce the material implant from a liquid polymer precursor applied directly on and around the site of the ocular defect.

By “polymer” is meant a molecule formed by the union of two or more monomers. A “monomer” is a repeating structure unit within a polymer. “Polymerization” is the bonding of two or more monomers to produce a polymer. For example, polymerization of ethylene forms a polyethylene chain, or polymerization of a monomer X and a monomer Y can yield a polymer with the repeating subunit X-Y. It will be appreciated that polymers can also be formed by the polymerization of more than two monomers and that two or more monomers can be present in unequal ratios in the resultant polymer. By “polymer precursor” is meant a molecule that is subsequently linked by polymerization to form a polymer, which is larger than the polymer precursor.

As discussed in greater detail below, polymerization can be achieved in various ways, such as by photopolymerization, autopolymerization, or physicochemical polymerization. The polymer precursor can itself be a polymer, such as, for example, poly(ethylene glycol). Alternatively, the polymer precursor can be a molecule other than a polymer, such as a protein, for example, albumin, collagen, gelatin, or other non-polymeric molecules.

The polymer precursor is usually present in the polymer formulation at a concentration in a range of about 0.01% to about 90%. The actual concentration varies with the polymer precursor used and its toxicology. Most polymer precursors are preferably used at a minimal concentration of about 5% because at lower concentrations it may be difficult to form a gel. However, by increasing the hydrophobicity of the ends of the polymer precursor, concentrations as low as about 1%, preferably about 3%, can be used to form a gel. High molecular weight precursors (i.e., greater than about 70,000 g/mol, preferably greater than about 100,000 g/mol), such as, for example, acrylated hyaluronic acid are preferably present at a concentration not greater than about 1%. See, for example, U.S. Pat. Nos. 5,801,033; 5,820,882; 5,626,863; and 5,614,587, incorporated herein by reference.

Transformation of the polymer precursor to a thin, gel-like coat can be accomplished in a number of ways, for example, by photochemical reactivity, by chemical reactivity, and/or by physicochemical response. When such a liquid-to-solid transition occurs directly upon the tissue surface, via any of the approaches described above, the resulting biomaterial implant adheres to the tissue surface. Liquid polymer precursor is applied over and around the ocular defect, covering the breached area of the defect and overlapping the unbreached area of the defect by an amount sufficient to maintain adhesion of the polymerized implant to the ocular surface. Typically, the polymerized implant extends over the unbreached area of the defect by about 0.1 mm to about 5 mm, and can extend over a substantial portion of the ocular surface if desired, up to the entire ocular surface. Preferably the polymerized implant extends over the unbreached area of the defect by about 0.5 mm to about 2 mm.

The transformation of polymer precursor into a gel-like coat can be achieved by photopolymerization of the polymer formulation. Photochemically activatable polymer precursors suitable for the methods of the invention include precursors comprising a water-soluble polymer as the central domain, such as, for example, poly(ethylene glycol) (PEG)-based polymers. PEG is a polymer of the formula HOCH₂ (CH₂OCH₂)ₙCH₂OH, wherein n is an integer giving rise to molecules ranging in molecular weight typically from about 200 g/mol to greater than about 75,000 g/mol, preferably between about 6,000 g/mol to about 35,000 g/mol. Some specific PEG molecules have a molecular weight of about 400, 1350, 3350, 4000, 6000, 8000, 18500, 20000, or 35000. PEG molecules having a molecular weight not specifically listed, but nonetheless within a range of about 200 g/mol to greater than about 75,000 g/mol are also contemplated. Lower molecular weight PEG formulations are referred to as short chain PEG formulations and typically have a molecular weight of about 4,000 g/mole or less. Higher molecular weight PEG formulations are referred to as long chain PEG formulations and have a molecular weight of greater than about 4,000 g/mol, preferably greater than about 8,000 g/mol, and can be greater than about 10,000 g/mol, and greater than about 20,000 g/mol. Preferably the long chain PEG formulations have a molecular weight in the range of about 7,000 g/mol to about 20,000 g/mol, with about 8,000 g/mol to about 10,000 g/mol being most preferred. One of ordinary skill in the art expects PEG molecules to be present in a distribution centered around the stated molecular weight, commonly as much as plus or minus about 20% of the stated molecular weight. Vendors often list the molecular weight of a PEG product as an average molecular weight (See, for example, the Sigma catalog; Sigma-Aldrich; St. Louis, Mo.).

Preferably the polymer precursors of the invention comprise reactive termini to allow for photopolymerization, such as, for example, free radical polymerizable termini. Examples of such reactive termini include acrylates and methacrylates, with acrylates being more preferred. Preferably the polymer precursor is a PEG diacrylate or tetracrylate.

Preferably the polymer precursor also comprises degradable regions of a molecular weight, relative to that of the water-soluble central domain, to be sufficiently small that the properties of the polymer precursor in solution, and the gel properties, are determined primarily by the central water-soluble chain. Typically the polymer precursor comprises about 0% to about 20%, preferably about 1% to about 10%, degradable regions. Examples of such degradable regions include, but are not limited to, hydrolytically labile oligomeric extensions, such as, for example, poly(a-hydroxy esters). Examples of poly(a-hydroxy esters) include poly(dl-lactic acid) (PLA), poly(glycolic acid) (PGA), poly (3-hydroxybutyric acid) (HBA), and polymers of ε-caprolactone. The hydrolytic susceptibility of some of the ester linkages is in the following order: glycolidyl>lactoyl>ε-caprolactyl.

In a preferred embodiment, the polymer precursor has the formula: Pₘ-Dₙ-Wₒ-Dₚ-P_{q}, wherein W is a water-soluble polymer; D is a degradable moiety; P is a photopolymerizable moiety; m and q are integers from 1 to about 10; o is an integer from 1 to about 100; and n and p are integers from 0 to about 120. W can be a linear polymer or a branched polymer. One of ordinary skill in the art would understand the formula provided above to include branched polymers having more than two termini and having degradable and/or photopolymerizable moieties on some or all of the termini of the branched polymer. A “degradable moiety” is an oligomeric compound that when integrated into a polymer precursor, creates within the polymer precursor a degradable region as described above. A “photopolymerizable moiety” is a moiety that allows the polymer precursor to polymerize upon exposure to light. Some wavelengths suitable for catalyzing polymerization are discussed in more detail below.

Typically, the values of m and q are varied so as to achieve the desired degree of cross-linking and rate of transition from liquid-to-gel upon polymerization. The values of n and p are varied so as to achieve a desirable percentage of the degradable moiety, preferably between about 0.1% to about 25% degradable moiety, with about 1% to about 10% being most preferred. One of ordinary skill in the art would know to vary the values for n and p according to the value of o and 20 the molecular weight of W in order to achieve this goal. Preferably m and q are integers from 1 to about 5, n and p are integers from 0 to about 10, and o is an integer from 1 to about 40. Alternatively, the polymer formulation can comprise in varying molar ratios polymer precursors having differing values for m, n, o, p and q so as to achieve a desirable percentage of the degradable moiety upon polymerization. For example, if W is a water soluble polymer having a molecular weight of at least 4,000 g/mol and o=1, n and p are integers from 0 to about 60, more preferably 30 from 0 to about 25, even more preferably 1 to about 15, with 1 to about 5 being most preferred. Preferably W is a PEG molecule having a molecular weight from about 200 g/mol to about 75,000 g/mol. Preferably, if W is a PEG molecule having a molecular weight greater than 4,000, o is an integer Preferably the polymer precursor comprises a PEG central chain with degradable regions and photopolymerizable end groups that terminate the degradable regions. The polymer precursors of the invention can be synthesized by methods known in the art (Sawhney et al., Macromolecules (1993) 26:581-587; Hill-West et al., Proc. Natl. Acad. Sci. USA (1994) 91:5967-5971) and described herein in Examples 1-3.

A preferred polymer chain comprises lactic acid, glycolic acid or epsilon-caproic acid in the degradable region D. Incorporation of oligolactic acid into the polymer will increase its hydrophobic content. The polymer's hydrophobic content, and hence its strength of adhesion, varies directly with its % oligolactic, oligoglycolic, or oligoepsilon-caproic acid content. PEG is used to initiate the ring-opening polymerization of dl lactide, ll lactide, glycolide, or epsilon caprolactone to an extent such that from about 0.1% to about 25%, preferably about 1% or 10%, of the mass of the polymer chain is comprised of oligolactic acid, oligoglycolic acid, or oligoepsilon-caproic acid. This ratio is controlled via the reaction stoichiometry: the polymerization, if performed on dry polymer precursor, will produce very little lactic acid, glycolic acid, or epsilon-caproic acid homopolymer.

Biocompatibility of various biodegradable polymers can easily be assessed as described in Example 6 by injecting rabbits intravitreally with a polymer formulation, photopolymerizing the polymer precursor, and observing the animal clinically or histologically for signs of intraocular inflammation or toxicity.

The polymer precursors can be photopolymerized to form cross-linked networks directly upon the retinal surface. In addition to the polymer precursors, the biodegradable polymer formulation can also comprise reagents to facilitate the photopolymerization process, such as at least one photoinitiator, and one or more co-catalysts, such as, for example, N-vinylpyrrolidone and triethanolamine. Preferably a nontoxic photoinitiator such as eosin Y photoiniator is used. Other initiators include 2,2-dimethoxy-2-phenylacetophenone and ethyl eosin. The polymerization process can be catalyzed by light in a variety of ways, including UV polymerization with a low intensity lamp emitting at about 365 nM, visible laser polymerization with an argon ion laser emitting at about 514 nM, visible illumination from a conventional endoilluminator used in vitreous surgery, and most preferably by illuminating with a lamp that emits light at a wavelength between 400-600 nM, such as, for example, a 1-kW Xe arc lamp. Illumination occurs over about 1-120 seconds, preferably less than about 30 seconds. Since the heat generated is low, photopolymerization can be carried out in direct contact with cells and tissues. Indeed, similar materials have been successfully utilized for the encapsulation of pancreatic islet cells and for the prevention of post-operative adhesion formation (Hill-West et al. Obstet Gynecol 83: 59-64 (1994).

Alternatively, the transformation of the polymer formulation into a gel-like coat can be achieved by autopolymerization of the polymer formulation. Auto-chemically reactive polymer gels may be formed by mixing two or more mutually reactive polymer precursors to result in a cross-linked polymer network. Usually, the polymer formulation comprises a first polymer precursor and a second polymer precursor, the first and second polymer precursors being mutually reactive. Preferably the first and second polymer precursors are present in about equimolar amounts. Typically, at least one of the reactive polymer precursors is a PEG-based polymer precursor. Preferably, both polymer precursors are PEG-based polymer precursors.

Suitable first polymer precursors include proteins, such as, for example, albumin, proteins derived from skin, connective tissue, or bone, such as collagen or gelatin, other fibrous proteins and other large proteins, tetra-amino PEG, copolymers of poly(N-vinyl pyrrolidone) containing an amino-containing co-monomer, animated hyaluronic acid, other polysaccharides, and other amines. Preferably the tetra-amino PEG has a molecular weight of at least about 3,000 g/mol, preferably more than about 6,000 g/mole, more preferably more than about 10,000 g/mol, and more preferably at least about 20,000 g/mol.

Suitable second polymer precursors include, but are not limited to, terminally-functionalized PEG, such as difunctionally activated forms of PEG. Some activating groups include epoxy groups, aldehydes, isocyanates, isothiocyanates, succinates, carbonates, propionates, etc. Examples of such forms of PEG include, but are not limited to, PEG di-succinimidyl glutarate (SG-PEG), PEG di-succinimidyl (S-PEG), PEG di-succinimidyl succinamide (SSA-PEG), PEG di-succinimidyl carbonate (SC-PEG), PEG di-propionaldehyde (A-PEG), PEG succinimidyl propionate, and PEG di-glycidyl ether (E-PEG) (U.S. Pat. No. 5,614,587) and other epoxy-derivatized PEG molecules, PEG nitrophenyl carbonate, PEG dialdehydes, PEG di-isocyanates, PEG di-isothiocyanates, and the like. Particularly preferred is a di-N-hydroxysuccinimidyl-activated dicarboxyl (PEG), such as a di-N-hydroxysuccinimidyl PEG. Other suitable difunctionally activated forms of PEG can be obtained from the Shearwater Polymers Catalog (see, for example, the “Electrophilically Activated” section of their website)

Preferred autochemically reactive polymer precursor pairs include (1) a tetra-amino PEG and a di-N-hydroxysuccinimidyl PEG; (2) a tetra-amino PEG and a di-succinimidyl carbonate PEG; (3) collagen, gelatin, or albumin and a di-N-hydroxysuccinimidyl PEG; (4) collagen, gelatin, or albumin and a di-succinimidyl carbonate PEG; and (5) other suitable autochemically reactive polymer pairs. Most preferred for the methods of the invention is the combination of a tetra-amino PEG and a di-N-10 hydroxysuccinimidyl PEG. If a di-N-hydroxysuccinimidyl active PEG is mixed with a di-amino PEG, a high molecular weight polymer results, but not a cross-linked hydrogel. However, if a di-N-hydroxysuccinimidyl activated PEG is mixed with a tetra-amino PEG, a cross-linked hydrogel network is formed, liberating only N-hydroxysuccinate as a reaction product. N-hydroxysuccinate is water-soluble and of very low toxicity. Preferably the di-N-hydroxysuccinimidyl PEG used in combination with a tetra-amino PEG is a di-N-hydroxysuccinimidyl activated succinate-terminated PEG. Di-N-hydroxy-succinimidyl activated glutarate-terminated PEG is less preferred because, when used in combination with a tetra-amino PEG, can produce ocular inflammation. These hydrogels can degrade by spontaneous hydrolysis at the linking group at the end of the polymer chain and can degrade within the protein backbone of a protein-containing gel. With gels formed from a PEG-containing first component and a PEG-containing second component, one can include a hydrolytically degradable oligolactic acid, oligoglycolic acid, or oligoepsilon-caproic acid domain, for example. Gels formed from protein-based, peptide-based, or polysaccharide-based precursors can also degrade under the enzymatic influences of the body.

Biocompatibility of various reactive polymer precursor pairs can easily be assessed as described in Example 7 by injecting a rabbit intravitreally with a mixture of the members of the polymer precursor pair, and observing the animal visually or histologically for signs of intraocular inflammation or toxicity.

The extent of incorporation into the gel phase can be optimized by manipulating various parameters, such as the pH of the reaction solution and the ratio of the first polymer precursor to the second polymer precursor. Typically, when PEG tetra-amine and di-N-hydroxy succinimidyl PEG are to be used, polymer precursors are separately reconstituted immediately before use in physiological saline at pH 8. They are mixed to yield a total final concentration of about 10% using an optimal ratio of molar amounts of each precursor, preferably equimolar. Given that reaction begins immediately after mixing, injection onto the retina is preferably performed immediately. The mixing is performed with two syringes and a connector. Alternatively, a syringe with two barrels can be used. Static mixture occurs on the tip of the syringe immediately before the polymer precursor solutions pass through a needle or cannula. The time between the initiation of mixing and injection is usually less than about 30 seconds. This can be achieved by positioning a 30 gauge cannula (or other suitable sized cannula, or a needle) attached to a syringe(s) containing polymer over the area to be treated prior to mixing the components.

Toward physicochemical transition, block copolymers of poly(ethylene glycol)-poly(propylene glycol)-poly (ethylene glycol), commonly referred to as Pluronics™, can be used to form polymer solutions that are liquid at 4° C. but gels at 37° C., permitting injection of the cold fluid with solidification to form a physicochemically cross-linked polymer network on the surface of the tissue. Other thermoreversible biocompatible biodegradable polymers are known. For example, Jeong et al., Nature (1997) 388:860-862, recently described copolymers of PEG and lactic acid that display favorable liquid-to-solid gelation transitions. Such materials can either be applied warm and fluid and allowed to cool in vivo into a gel form, or can be applied cool and fluid and allowed to warm in vivo into a gel form, depending upon the physiochemical characteristics of the gel and its precursor.

Polymers that display a physicochemical response to stimuli have been explored as potential drug-delivery systems. Stimuli studied to date include chemical substances and changes in temperature, pH and electric field. Homopolymers or copolymers of N-isopropylacrylamide and poly(eythlene oxide)-poly(propylene oxide)-poly (ethylene oxide) (known as poloxomers) are typical examples of thermosensitive polymers, but their use in drug delivery is problematic because they are toxic and non-biodegradable. Biodegradable polymers used for drug delivery to date have mostly been in the form of injectable microspheres or implant systems, which require complicated fabrication processes using organic solvents. Such systems have the disadvantage that the use of organic solvents can cause denaturation when protein drugs are to be encapsulated. Furthermore, the solid form requires surgical insertion, which often results in tissue irritation and damage. The methods of the invention involve the synthesis of a thermosensitive, biodegradable hydrogel consisting of polymer precursor blocks of poly(ethylene oxide) and poly(L-lactic acid). Aqueous solutions of these polymer precursors exhibit temperature-dependent reversible gel-sol transitions. By “sol” is meant a polymer precursor solution which is more liquid than solid. By “gel” is meant a polymer solution which is more solid than liquid. The hydrogel can be loaded in an aqueous phase at an elevated temperature (around 45 degrees C.), where they form a sol. In this form, the polymer is injectable. On subcutaneous injection and subsequent rapid cooling to body temperature, the loaded copolymer forms a gel.

The polymer formulations described above are applied in a manner consistent with the surgical procedure as a whole. Typically, the ocular tissue is prepared for subsequent administration of the polymer. For example, in posterior capsule opacification, after removal of the cataract, the anterior segment may be filled with a gas bubble, and the polymer is then placed over the posterior capsule.

The polymer formulation is then applied to the ocular tissue, such as is described above. Polymerization is effected as discussed above, such as chemical or light-induced polymerization. Usually at least-about 1 second to about five minutes or longer is allowed to pass to ensure complete polymerization has occurred, and preferably the delay is less 30 seconds.

Another aspect of the invention is a method for management of an ocular defect in an animal, comprising applying a non-toxic, biodegradable polymer formulation to the ocular tissue of the animal over and around the ocular defect, and transforming the polymer formulation into a gel-like coat. As discussed above, closure (or sealing) of the ocular defect reduces fluid leakage into undesirable regions of the eye.

Yet another aspect of the invention is a method for the prevention of an ocular defect, comprising applying a non-toxic, biodegradable polymer formulation to the ocular tissue of an animal in need thereof. Preferably, the polymer formulation is applied to at least about 25% of the ocular tissue surrounding the ocular defect, preferably to more than about 50% and applications to more than about 75% of the ocular tissue to the entire ocular tissue are most preferred. In a preferred embodiment, a polymer precursor solution comprising at least one photoinitiator is applied to the ocular tissue around the ocular defect. Polymerization is then effected by any of the methods described above to close the ocular defect. The eye is then filled with a solution containing at least one photoinitiator but no polymer precursor to coat the ocular tissue. Excess photoinitiator is drained from the eye. Next, polymer precursor solution that does not contain photoinitiator is applied to the remainder of the ocular tissue and polymerization is again effected. The polymerization reaction results in a thin, transparent gel where the polymer precursor contacts the photoinitiator, but not in areas free of photoinitiator. This results in the formation of a gel only on the ocular tissue. The eye is once again filled with fluid. Unpolymerized precursors are then irrigated from the eye. The adherent polymer biodegrades over about a 2-10 week period. The polymerized gel overlying the ocular defect both closes the ocular defect and prevents adherence of scar tissue that could cause subsequent problems. Another embodiment omits the initial step of applying a polymer precursor solution containing photoinitiator directly to the defect.

A further aspect of the invention is the use of at least one non-toxic, biodegradable polymer precursor for the preparation of a pharmaceutical composition for treating an ocular defect in a mammal. Suitable polymer precursors and other components of the pharmaceutical composition are discussed in detail above in the sections describing the components of suitable polymer formulations. Additional components can include any other reagents that catalyze polymerization of the polymer precursor, pharmaceutically suitable delivery vehicles for ocular administration, such as for delivery to the interior of the eye, and any other pharmaceutically acceptable additives.

The invention also provides articles of manufacture for use in a mammal with a non-toxic biodegradable polymer. In one embodiment, the article of manufacture comprises a first container comprising a polymer precursor of the formula Pₘ-Dₙ-Wₒ-Dₚ-P_{q}, wherein W is a water-soluble polymer; D is a degradable moiety; P is a photopolymerizable moiety; m and q are integers from 1 to about 10; o is an integer from 1 to about 100; and n and p are integers from 0 to about 120. The first container can optionally contain at least one photoinitiator and can also optionally contain at least one co-catalyst. Where the first container does contain a photoinitiator in addition to the polymer precursor, the article of manufacture can optionally contain a second container comprising polymer precursor but no photoinitiator. The article of manufacture can optionally contain a third container comprising a photoinitiator solution but no polymer precursor. An article of manufacture comprising all three containers or just the second and third containers are useful for preventing at least some ocular diseases or conditions as described above. The article of manufacture preferably further comprises instructions for use according to the methods described above involving photopolymerization.

In another embodiment, the article of manufacture comprises a first container comprising a first polymer precursor and a second container comprising a second polymer precursor, the first and second polymer precursors being mutually reactive. The first and second polymer precursors can be present in the container in admixture with a pharmaceutically suitable vehicle for delivery to the interior of the eye. Alternatively, any such vehicle can be added separately, if necessary, for example, to reconstitute the polymers. Suitable first and second polymer precursors are any of those polymer precursor pairs discussed above that can autopolymerize. Preferably the first polymer precursor is albumin, collagen or gelatin, and the second polymer precursor is a terminally-functionalized poly(ethyl ene glycol) (PEG). Typically, the first and second containers are separate syringes or are separate barrels of a single syringe having static mixture device at the tip of the syringe, and can also be vials or other cylindrical containers, such as, for example, a segment of tubing. The article of manufacture can further comprise printed instructions for a method for providing a PEG-based polymer to an ocular defect by combining the first and second polymer precursors immediately before applying to the retinal surface of the mammal over and around the ocular defect. Usually, the first and second polymer precursors are combined by extruding from each container simultaneously into and through a connector onto the retinal surface. Suitable connectors are any structures that permit mixing of the first and second polymer precursors immediately before application to the ocular tissue surface, such as, for example, a structure that is Y-shaped and comprises two tubular segments, each of which fits over an aperture in each container, and which are united into a single tubular segment.

### E. PEG-Based Polymers as a Barrier-Intraocular Hypotony

In one embodiment of the present invention, the PEG-based polymer provides a barrier function for at least one ocular defect. In a specific embodiment of the present invention, the polymer may act as a sealant and as a barrier. In another specific embodiment, the barrier may be a mechanical barrier and/or may act as a barrier to prevent, inhibit, and/or reduce cellular adhesion and/or proliferation of an ocular defect. In some embodiments there is a method of forming at least one barrier to adhesion and/or of preventing cellular adhesion and proliferation in the eye of a mammal by applying at least one polymer as described herein to an ocular defect.

In a particular embodiment, the present invention is directed to improving eye vision by increasing the intraocular hypotony in the eye, which in some embodiments is through utilization of the PEG-based polymers of the present invention. In a specific embodiment, the method of forming at least one barrier to adhesion and/or of preventing cellular adhesion and proliferation in the eye of a mammal comprises increasing the intraocular hypotony by applying at least one PEG-based polymer as described herein.

In a specific embodiment, the eye has insufficient intraocular pressure. In specific embodiments, the pressure is less than about 5 mmHg. The invention achieves this goal in a novel manner by reducing outflow of aqueous from the eye, as opposed to known methods that increase production of aqueous fluid.

A skilled artisan recognizes aqueous is formed in the ciliary body behind the iris, and it flows through the pupillary space into the anterior chamber. From this point, the fluid travels into the angle structures and drains from the eye. As the aqueous fluid leaves the angle, it passes through a filter referred to as the trabecular meshwork, then traveling through the Channel of Schlem, a tiny channel in the sclera. The aqueous flows into other tiny vessels and eventually leads into the blood vessels of the eye.

In specific embodiments, the methods involve blocking at least partially flow of aqueous from the angle, and particularly through the trabecular meshwork. In specific embodiments, this is achieved by inserting or applying a biocompatible polymer to reduce the flow. In further specific embodiments, a biocompatible photopolymerizable polyethylene glycol-based polymer obstructs the trabecular meshwork, thereby reducing aqueous egress from the anterior chamber angle. A PEG-based polymer comprises an ethylene-glycol unit covalently attached to another ethylene-glycol unit and/or an acrylate, an ester, or a carbonate. One skilled in the art is aware of methods to determine appropriate structures thereof based on such factors as solubility of the unit. In specific embodiments, the polymer has biocompatibility, biodegradability, mutual reactivity, and other desirable properties well known to a skilled artisan.

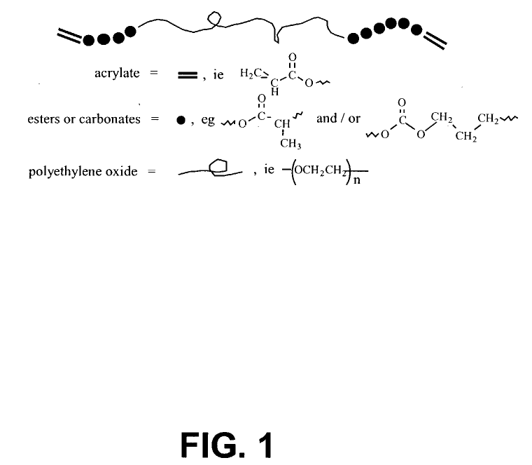

A non-limiting example of a polymer of the present invention that may be used as a suitable formulation is an “aqueous solution of a copolymer of poly(ethylene glycol)-cotrimethylene carbonate-co-lactide (M, 20,000) with acrylated end groups (see FIG. 1). The sealant solution was used as a 5% to 10% (w/w) solution in triethanolamine (90 mM)-buffered saline with eosin Y (20 ppm) added as a photoinitiator” (Alleyene et al., 1998).

A schematic diagram of a photochemically-reactive polymer is illustrated in FIG. 1. A skilled artisan is aware that polyethylene oxide is poly(ethyleneglycol) of molecular weight greater than about 20,000 Daltons (Da).

In a specific embodiment of the present invention, the procedure is performed in the following exemplary manner. In a specific embodiment, topical anesthesia is applied to control pain and/or discomfort prior to commencing the method. For treating intraocular hypotony, aqueous fluid is withdrawn from the anterior chamber with a needle, such as through paracentesis. A device is applied or a procedure is performed to facilitate directing the material to be injected into the angle. In a specific embodiment, an air bubble is injected into the anterior chamber via the same paracentesis site as the fluid was withdrawn to facilitate direction of the injection.

A polymer precursor is then injected into the anterior chamber and directed toward the angle; it is preferably injected around either portions of (<360 degrees), or the entire anterior chamber angle (360 degrees) via one or more paracentesis sites. A skilled artisan recognizes that the large central bubble helps to direct the material into the angle. Polymerization then occurs in situ, such as in an automatic manner, or through externally applied manipulation, such as by irradiation. In a specific embodiment, photopolymerization is performed by applying a source of visible light directly over the peripheral cornea where the polymer precursor has been placed. In an alternative embodiment to using photopolymers for methods of the present invention, autopolymerizing formulations are used to reduce aqueous outflow. These formulations polymerize in situ without the need for photo-stimulation. In the case of photopolymerization, the light is applied continuously, such as for at least about 30 seconds to 120 seconds, and up to 6 minutes or more in some embodiments, and preferably in about a 360-degree fashion. In some embodiments, the duration of stimulation for polymerization is for shorter than 30 seconds, depending on the polymer formulation used (% PEG).

The device or procedure that facilitated direction of the material into the angle is then preferably removed. For example, the central air bubble may then be aspirated with a needle. Once the polymer is polymerized, it adheres to, for example, the trabecular meshwork and/or adjacent structures (such as the peripheral cornea or iris). In this position, it retards aqueous outflow and intraocular pressure is increased. Until the gel biodegrades, it increases intraocular pressure. Once degraded, additional polymer can be injected and polymerized, such as by a similar method.

3. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Synthesis of 6 KD PEG Polymer Precursor

A PEG-co-poly(“-hydroxy acid) copolymer is synthesized. A total of 30 g of dry PEG 6K, 3.60 g of dl-lactide (5 mol dl-lactide/mol of PEG), and 15 mg of stannous octanoate are charged into a 100-mL round-bottomed flask under a nitrogen atmosphere. The reaction mixture is stirred under vacuum at 200° C. for 4 h and at 160° C. for 2 h and is subsequently cooled to room temperature. The resulting copolymer is dissolved in dichloromethane, precipitated in anhydrous ether, filtered, and dried. The α- and ω-hydroxyl end groups of PEGS with various molecular weights are used as ring-opening reagents to initiate the polymerization of either dl-lactide or glycolide to similarly form several other copolymers.

The copolymers are end-capped with acrylate groups to form a polymerizable polymer precursor. A total of 30 g of the intermediate copolymer is dissolved in 300 mL of dichloromethane in a 500-mL round-bottomed flask and is cooled to 0° C. in an ice bath. A total of 1.31 mL of triethylamine and 1.58 mL of acryloyl chloride are added to the flask, and the reaction mixture is sitrred for 12 h at 0° C. and 12 h at room temperature. The reaction mixture is filtered to remove triethanolamine hydrochloride, and the polymer precursor is obtained by pouring the filtrate in a large excess of dry diethyl ether. It is further purified by dissolution and reprecipitation once using dichloromethane and hexane, respectively. Finally, it is dried at 70° C. under vacuum for 1 day.

### EXAMPLE 2

### Synthesis of 10,000-Da PEG Polymer Precursor

A macromolecular precursor is synthesized that consists of a central chain of poly(ethylene glycol) (PEG) with flanking regions of lactic acid oligomer and tetra-acrylate termini. The precursor is synthesized by dissolving 50 g of 10,000-Da PEG (Sigma) in toluene (Mallinckrodt, ACS grade) and refluxing under argon for 1 hour. 4.5 g of dl-lactide (Aldrich) and 50 μl of 50% (vol/vol) stannous octanoate (ICN) in toluene are added. The solution is refluxed under argon for 16 hours to achieve an average of five lactic acid groups per end, as estimated by proton NMR. The solution is cooled to about 20° C., and the polymer is precipitated with hexane (Mallinckrodt, ACS grade), filtered, washed, and dried. This polymer is dissolved in tetrahydrofuran (Mallinckrodt, ACS grade) under argon and cooled to about 15° C. 5.23 ml of triethylamine (Aldrich) and 3 ml of acryloyl chloride (Aldrich) are added to the mixture while bubbling argon through the solution. The mixture is then refluxed under argon for 24 hours. Triethylamine hydrochloride precipitate is removed by filtration. The macromolecular precursor is precipitated with hexane, filtered, washed, and dried. The precursor is stored at 0° C. under argon until use.

### EXAMPLE 3

### Synthesis of PEG Diacrylates of Various Molecular

Weights

PEG diacrylates of various molecular weights are synthesized as described in Cruise et al., Biomaterials 19:1287-1294 (1998). All solvents used in the synthesis are reagent grade or better and the reactants are used as received.

Fifty grams of PEG diol (Union Carbide) with a molecular weight of either 1350 (2K), 3350 (4K), 8000 (8K) or 20,000 (20K) were dissolved in 750 ml of benzene (Fisher) and water was removed by azeotropically distilling 250 ml of benzene. Triethylamine (Aldrich), in four fold molar excess based on PEG diol end groups, is added to the PEG solution at room temperature. Acryloyl chloride (Aldrich), in four fold molar excess based on PEG diol end groups, is added dropwise to the PEG solution to form acrylate diesters of PEG. The mixture is stirred overnight at 35° C. under argon. The insoluble triethylamine salts formed during the reaction are removed by filtration and the PEG diacrylate product is precipitated by the addition of 1.4 liters of diethyl ether (Fisher) chilled to 4° C. The PEG diacrylate precipitate is collected on a fritted funnel, redissolved in 100 ml of benzene, and reprecipitated with 1.4 liters of chilled diethyl ether twice more. The polymer is dried 24 h in a vacuum oven at 35° C.

PEG diacrylates are analyzed using nuclear magnetic resonance (NMR) spectroscopy and gel permeation chromatography (GPC). The degree of substitution of the PEG terminal alcohol for acrylate is determined using the NMR spectrum of PEG diacrylates and the method of Dust et al., Macromolecules 23:3743-3746 (1990), which compares the ratio of the integration from the PEG backbone (−3.5 ppm) and the acrylate peaks (˜5.8-6.4 ppm) to the known PEG weight average molecular weight. The extent of acrylation substitution is calculated using the following formula: % acrylation={PEG molecular weight}/{(integral of PEG backbone)/[(integral of acrylates)/6]/4×44}.

### EXAMPLE 4

### Visible Laser Polymerization of 10,000-Da PEG

Polymer Precursor

The tissue is incubated in 1 mM eosin Y (Sigma), a nontoxic photoinitiator, in Hepes-buffered saline (10 mM, pH 7.4) for 1 minute to adsorb the photoinitiator onto the surface of the tissue. The tissue is then rinsed twice in Hepes-buffered saline and infused with a 23% solution of the macromolecular precursor that also contains 100 mM triethanolamine (Aldrich) and 0.15% N-vinylpyrrolidone (Aldrich). The tissue is illuminated using an argon ion laser (514 nm, 70 mW/cm2, 2-s exposure; American Laser, Salt Lake City) to convert the liquid precursor to a hydrogel on the surface of the tissue.

### EXAMPLE 5

### Polymerization of 10,000-Da PEG Polymer Precursor with Emitted Light Between 400 nm and 600 nm

The tissue is contacted with 1 mM eosin Y in Hepes-buffered saline, which is allowed to adsorb to the tissue for 1 minute. The eosin Y is withdrawn, and the tissue is rinsed twice with saline. The tissue is then contacted with a 23% solution of the precursor that also contains 100 mM triethanolamine and 0.15% N-vinylpyrrolidone. The tissue is then externally illuminated with a 1-kW Xe arc lamp that emits light between 400 and 600 nm (Optomed, Austin, Tex.) at an irradiance of 35 mW/cm². Illumination times are between 2 and 15 s.

### EXAMPLE 6

### Assessing Biocompatibility of Photochemically Reactive Polymer Formulations

Dutch Banded Rabbits are given general anesthesia with an intramuscular injection of xylazine and ketamine. Two Dutch Rabbits eyes are injected intravitreally with 100 μl of a mixture of a photochemically reactive polymer precursor, N-vinylpyrrolidone (1500 ppm), triethanolamine (20 mM), and eosin Y photoinitiator (10 μM) in a balanced saline solution. An external, hand-held Xenon arc light source (400-600 nm) is used to irradiate the globe of the eye for 1 minute.

The eyes are examined clinically with slit lamp and indirect ophthalmoscopy at days 1 and 5 post-injection for media opacity or other signs of ocular toxicity. Rabbits are then sacrificed on day 5 and the eyes are examined for histologic evidence of intraocular inflammation or toxicity.

Long Chain PEG (20,000 g/mol)

At days 1 and 5 post-injection of a polymer formulation containing 23% long chain PEG (20,000 g/mol), no media opacity or other signs of ocular toxicity were evident and the fundus was clearly visible in both eyes. The rabbits were sacrificed on day 5 and the eyes were processed for light microscopy. The animals showed no histologic evidence of intraocular inflammation or toxicity. The iridocilliary processes showed none of the inflammatory processes evident in rabbit eyes injected with di-N-hydroxy succinimidyl activated glutarate-terminated PEG (Example 7). There was no fibrinoid reaction in the vitreous cavity. There was no inflammatory process evident in the retina or in the vitreous cavity.

Short Chain PEG (4,000 g/mol)

Rabbits were treated as described above, except that a retinal break was created as described below in Example 8. The animals were examined at 1 and 7 days after injection of a polymer formulation containing 23% short chain PEG (4,000 g/mol) by penlight and indirect ophthalmoscopy. Severe intraocular inflammation was evident in both treated eyes. A fibrinous pupillary membrane obscured the pupil of one eye and no view of the fundus was possible in either treated eye.

### EXAMPLE 7

### Assessing Biocompatibility of Autochemically Reactive Polymer Formulations

Dutch Banded Rabbits are given general anesthesia with an intramuscular injection xylazine and ketamine. Autochemically reactive polymer precursors are mixed in a balanced saline solution and 100 μl is injected intravitreally using a 27 gauge needle on a tuberculin syringe. A gel is allowed to form.

The eyes are examined at days 1 and 5 for signs of intraocular inflammation and opacification of the ocular media. The rabbits are sacrificed on day 5 and the eyes are examined for histological evidence of intraocular inflammation or toxicity.

Di-N-hydroxysuccinimidyl Activated Glutarate-Terminated PEG

PEG tetra-amine (molecular weight 20,000 g/mol) and di-N-hydroxysuccinimidyl activated glutarate-terminated PEG (molecular weight 3,500 g/mol) were mixed to yield a polymer formulation containing 11.5% of each polymer precursor and injected intravitreally. At days 1 and 5 post-injection, severe intraocular inflammation and opacification of the ocular media were evident. The pupil was obscured and no view of the fundus was possible. The rabbits were sacrificed on day 5 and the eyes were processed for light microscopy. Both eyes showed marked inflammatory cell infiltration of the uveal tract and vitreous cavity. The iridocilliary processes were haemmorhagic and edematous. A marked suppurative reaction with multiple eosinophilic polymorphonucleocytes was observed. A marked fibrinoid reaction was visible in the vitreous cavity. A subretinal inflammatory process was evident, with multiple eosinophilic polymorphonucleocytes that extended into the vitreous cavity. The inflammatory processes also extended into the anterior chamber.

Di-N-hydroxysuccinimidyl Activated Succinate-Terminated PEG

PEG tetra-amine (molecular weight 20,000 g/mol) and di-N-hydroxysuccinimidyl activated succinate-terminated PEG (molecular weight 3,500 g/mol) were mixed to yield a polymer formulation containing 11.5% of each polymer precursor and injected intravitreally. At days 1 and 5 post-injection, no media opacity or other signs of ocular toxicity were evident. The rabbits were sacrificed on day 5 and the eyes were processed for light microscopy. The rabbit eyes showed no histologic evidence of intraocular inflammation or toxicity. The iridocilliary processes showed none of the inflammatory processes evident in rabbit eyes injected with glutarate-terminated PEG as described above. There was no fibrinoid reaction in the vitreous cavity. There was no inflammatory process evident in the retina or in the vitreous cavity.

### EXAMPLE 8

### Assessing Adherent Properties of Polymer Implant

Two New Zealand White Rabbits are given general anesthesia with an intramuscular injection of xylazine and ketamine. They are then pre-treated with cryotherapy behind the nasal and temporal limbus in the ora serrata region under direct visualization. Two weeks later, using sterile technique, the animals undergo vitrectomy and lensectomy. Endodiathermy is then used to create an approximate 1 disc diameter retinal break just superior to the medullary wing. Balanced saline solution is injected into the subretinal space using a 30 gauge cannula to create a localized retinal detachment. Fluid-gas exchange is then performed, and the retina is flattened. The polymer formulation is applied over the retinal break using a 30 gauge cannula. The fiberoptic endo-illuminator of the Premier Vitrector (Storz Instruments) is then used to irradiate the mixture for 1 minute, causing a thin, transparent polymerized gel to form over the retinal break. The eyes are then refilled with balanced saline solution. Attempts are made to displace the gel with the fiberoptic illuminator tip and the 30 gauge cannula.

Short Chain PEG

Short chain PEG diacrylate (molecular weight 4000 g/mol, ca. 10% concentration), N-vinylpyrrolidone (1500 ppm), and triethanolamine (20 mM) precursors were mixed with an eosin Y photoinitiator (10 μM) and applied over the retinal break. The polymer remained adherent to the hole and surrounding retina. Thus, it is possible to precisely apply the polymer precursor solution under gas, polymerize it with visible light, and form an adherent gel over the hole that resists mechanical displacement.

### EXAMPLE 9

### Assessing Rate of Degradation of Polymer Implant

The duration of presence of non-toxic hydrogels on the retina is determined by incorporating commercially available 1 μM diameter fluorescence polymer beads (Polysciences) in the hydrogel precursor and thus in the hydrogel. This fluorescence can readily be observed in the eye non-invasively by the same type of fluorescence biomicroscopy commonly used to visualize the eyes of human patients given fluorescein. Eighteen Dutch Banded rabbits are given general anesthesia with an intramuscular injection of xylazine and ketamine. The right eyes are treated with cryotherapy behind the nasal and temporal limbus in the ora serrata region under direct visualization. Two weeks later, the animals are again given general anesthesia with an intramuscular injection of xylazine and ketamine. Lensectomy and vitrectomy are performed on the right eyes. A bent 30 gauge needle or vitrector is then used to create an approximately 1 disc diameter retinal break just superior to the medullary wing. Balanced saline solution is injected into the subretinal space using a 30 gauge cannula to create a localized retinal detachment. Fluid-gas exchange is then performed, and the retina is flattened. For example, the rabbits are divided into 3 groups of 6 rabbits each and given the treatments outlined below:

GROUP 1: 1% oligolactic acid photochemically reactive polymer

GROUP 2: 10% oligolactic acid photochemically reactive polymer

GROUP 3: Auto-chemically reactive polymer

All rabbits undergo vitrectomy, lensectomy, creation of a retinal break and detachment as described above. Fluid-gas exchange is then performed and laser photocoagulation applied around the retinal break in customary fashion. In each group of rabbits one of the hydrogel formulations and incorporated fluorescence polymer beads (Polysciences) is injected over and around the retinal break using a 30 gauge cannula. In the case of hotochemical hydrogels, the fiberoptic endo-illuminator is used to irradiate the mixture for 1 minute to form an adherent gel overlying the retinal hole. The eyes are filled with balanced saline solution, sclerotomies and conjunctiva are closed, and a subconjunctival injection of gentamycin is given. On post-operative days 1, 3, 7, 14, 21, and 28 the rabbits are examined by fluorescence biomicroscopy to determine whether polymer remains adherent to the retina. Because a chorioretinal adhesion may take up to 2 weeks to reach maximal strength, polymer formulations should ideally remain adherent to the retina for at least this amount of time but not more than 4 weeks. The animals are sacrificed after 28 days and the eyes are examined histologically.

### EXAMPLE 10

### Sealants

New Uses: Generally

As described in U.S. Pat. No. 6,149,931 (incorporated herein in by reference in its entirety), certain polymer compositions can be applied to mammalian tissue resulting in the formation, in situ, of a bioerodible (biodegradable) polymer. The polymer so created has specific properties, such as sealing ability and adhesion prevention. However, over a certain amount of time, the polymer is safely bioeroded (biodegraded).

Sealants: General Embodiments

Bioerodible (biodegradable) polymers are used in the instant invention in the prevention of egress of ocular fluid from inside the eye to the outside. In the eye, aqueous and vitreous fluids are under pressure, intraocular pressure (“IOP”). The IOP ranges between about 9-21 mmHg in normal individuals. This pressure is exerted upon the polymer sealant. To be effective, the polymer sealant must withstand this pressure without leaking. This type of use was not taught or indicated from earlier uses, including those of U.S. Pat. No. 6,149,931, because, with that patent, for example, the sealant existed within the eye itself and IOP was equal on either side of the sealant. In a particular embodiment of the present invention, the IOP is unequal on either side of the polymer, at least for some period of time.

Sealants: Filtration Surgery

In filtration surgery a protected opening in the eye is created to normalize intraocular pressure. The opening is covered by a sutured conjunctival incision. If the closure of conjunctiva is not water tight, leakage can occur. This results in a pressure that is too low (hypotony) and increases the risk of infection, choroidal hemorrhage, and a flat anterior chamber. PEG based polymer formulations (photo-polymerizing or chemical polymerizing) are applied to the conjunctival incision either alone or in conjunction with a suture based closure to prevent leakage. After about 2-16 weeks the PEG based sealant biodegrades. Similar applications to a glaucoma filtration bleb are performed in the post-operative period to seal a leaking bleb or a conjunctival buttonhole.

Sealants: Treatment of Glaucoma

On a patient, a standard limbal or formix-based trabeculectomy is performed on an eye with glaucoma refractory to medical management. Closure of the conjunctival incision is performed with a running suture. After tying the suture, a 1 cc syringe containing photopolymerizable PEG based polymer formulation is used to coat the wound via a 23 gauge cannula. After application, a xenon arc illuminator or endoilluminator is used to polymerize the formulation and seal the conjunctival incision. Three or four weeks later, wound healing has closed the wound and the polymer biodegrades.

Sealants: Corneal Surgery

In corneal surgery leakage of acqueous humor occurs across an inadequately sutured wound. This may occur following trauma, corneal transplantation, or perforating infectious and non-infectious processes. PEG based polymer formulations (photo-polymerizing or chemical polymerizing) are applied to the leaking corneal wound either alone or in conjunction with a suture based closure to prevent leakage. After 2-16 weeks the PEG based sealant biodegrades. No leakage occurs because fibrous proliferating has sealed the wound.

Sealants: Treatment of Corneal Injury

On a patient that suffered a penetrating corneal injury with a sharp metal object, having therefrom a stellate laceration of the cornea, microsurgical wound closure with interrupted 10-0 nylon sutures is performed. However, the wound has a persistent leak of aqueous humor. A 1 cc syringe containing photopolymerizable or chemical polymerizable PEG based polymer formulation is used to coat the wound via a 23 gauge cannula. After application, a xenon arc illuminator or vitreous endoilluminator is used to polymerize the formulation (if photopolymerizable) and seal the corneal laceration. Six to eight weeks later, wound healing has closed the corneal wound and the polymer biodegrades.

Sealants, Vitreoretinal Surgery:

In vitreoretinal surgery, sclerotomies are generally closed with sutures. As an alternative, PEG based polymer formulations (photo-polymerizing or chemical polymerizing) are applied to the scleral incision to achieve closure without the use of sutures. After about 2-16 weeks the PEG based sealant biodegrades.

Sealants: Vitrectomy

At the conclusion of a vitrectomy on a patient, the plugs and infusion cannula are removed successively from each sclerotomy. Prolapsed vitreous issuing through each sclerotomy is excised. Next, a 1 cc syringe containing photopolymerizable PEG based polymer formulation is used to seal the sclerotomy delivering a small aliquot via a 23 gauage cannula. After application, a xenon arc illuminator or endoilluminator is used to polymerize the formulation and seal the scleral incision. Three or four weeks later, wound healing has closed the wound and the polymer biodegrades.

### EXAMPLE 11

### Barriers to Adhesion

In filtration surgery, one of the leading causes of failure in scarring of the filtration site and bleb. To prevent or lessen formation of post-operative adhesions PEG based polymer formulations (photo-polymerizable or chemical polymerizing) are applied to the apposing tissue surfaces during surgery. Because the biodegradable polymers prevent cellular and protein adherence, adhesion formation is diminished compared to untreated patients. After about 2-16 weeks the PEG based sealant biodegrades. Because of the adhesion protection offered during the acute post-operative period, adhesion formation after polymer biodegradation is minimal.

Barriers: Trabeculectomy

In a standard trabeculectomy procedure, both the outside and inside surfaces of the scleral flap as well as the scleral margins surrounding the excised trabecular segment are coated using a 1 cc syringe containing photopolymerizable or chemical polymerizing PEG based polymer formulation delivered via a 23 gauge cannula. The undersurface of the conjunctival flap (with or without attached tenon's capsule) is also coated with a thin layer in the same fashion with the polymer formulation. After applying, a xenon arc illuminator or endoilluminator is used to polymerize the formulation (if photopolymerizable) and form a thin coat covering these tissue surfaces. Four to six weeks later, after the acute inflammatory response has subsided, the polymer biodegrades.

Barriers: Conjunctival Cicatricial Disease

In conjunctival, cicatricial disease, such as Stevens Johnson Syndrome, adhesions (symblepharon) form between the palpebral and bulbar conjunctival surfaces. To prevent such adhesion formation, PEG based polymer formulations (photo-polymerizing or chemical polymerizing) are applied to the bulbar and palpebral conjunctival surfaces. These biodegradable formulations bioreode in 1-24 weeks. Repeated applications are used if the inflammatory process persists. By preventing adhesions formation, the biodegradable polymer formulations minimize damage to the ocular surface.

Barriers: Acute Conjunctival Inflammation and Early Symblepharon

A patient with acute conjunctival inflammation and early symblepharon undergoes glass rod lysis of the symblepharon. Then, the bulbar and palpebral surfaces of the conjunctiva are coated using a 1 cc syringe containing photopolymerizable or chemical polymerizing PEG based polymer formulation delivered via a 23 gauge cannula. After applying, a xenon arc illuminator or endoilluminator is used to polymerize the formulation (if photopolymerizable) and form a thin coat covering these tissues surfaces. Four to six weeks later, after the acute inflammatory response has subsided, the polymer biodegrades.

Barriers: Strabismus or Scleral Buckling Surgery

In strabismus or scleral buckling surgery, adhesions commonly form between the extraocular muscles and adjacent surfaces (e.g. tenon's capsule, sclera, scleral buckle). These adhesions impair ocular motility and may cause double vision (diplopia). To prevent such adhesion formation, PEG based polymer formulations (photo-polymerizing or chemical polymerizing) are applied to exposed extraocular muscles and adjacent tissue or prosthetic surfaces. These biodegradable formulations bioreode in 2-16 weeks, after the acute post-operative period has passed and stimuli to adhesion formation are mitigated.

Barriers: Lateral Rectus Recession

On a patient, a standard lateral rectus recession of about 7 mm is performed. Prior to suturing the recessed muscles to the sclera, the muscle and adjacen scleral surfaces are coated using a 1 cc syringe containing photopolymerizable or chemical polymerizing PEG based polymer formulation delivered via a 23 gauge cannula. The undersurface of tenon's capsule is also coated in the same fashion with the polymer formulation. After applying, a xenon arc illuminator or endoilluminator is used to polymerize the formulation (if photopolymerizable) and form a thin coat covering these issues surfaces. Four to six weeks later, after the acute inflammatory response has subsided, the polymer biodegrades.

Barriers: Corneal Transplantation Surgery

In corneal transplantation surgery, endothelial graft rejection is associated with binding of white blood cells and proteins to the endothelial surface of the cornea. To prevent such rejections, PEG based polymer formulations (photo-polymerizing or chemical polymerizing) are applied to the endothelial surface of the donor cornea prior to suturing to the host during keratoplasty surgery. Because the biodegradable polymers prevent cellular and protein adherence, cellular and protein adhesion is diminished compared to untreated patients. After 2-24 weeks the PEG based sealant biodegrades. Because of the barrier to cellular and protein adhesion provided by the biodegradable polymers during the post-operative period, likelihood of graft rejection is minmized.

Barriers: Keratoplasty

During a standard keratoplasty procedure on a patient, the donor cornea is trephined and placed epithelial side down. Using a 1 cc syringe containing photopolymerizable or chemical polymerizing PEG based polymer formulation delivered via a 23 gauge cannula, the endothelium is coated with a thin layer of polymer formulation. After applying, a xenon arc illuminator or endoilluminator is used to polymerize the formulation (if photopolymerizable) and form a thin coat covering the endothelium. Eight to twelve weeks later, after the acute inflammatory response has subsided, the polymer biodegrades.

Barriers to Adhesion, After Cataract Surgery

After cataract surgery, lens epithelial cells frequently migrate over the posterior lens capsule and cause posterior capsule opacification. To prevent such opacification, PEG based polymer formulations (photo-polymerizing or chemical polymerizing) are applied to the posterior capsule prior to implantation of the intraocular lens. These biodegrable formulations bioreode in 6-48 months, after the biological stimuli prompting posterior capsule opacification subside.

### EXAMPLE 12

### Polymerization

A standard phacoemulsification procedure is performed on a patient. Prior to insertion of the IOL, the capsular bag and anterior chamber are filled with sterile air. Then, the posterior lens capsule is coated using a 1 cc syringe containing photopolymerizable or chemical polymerizing PEG based polymer formulation delivered via a 23 gauge cannula. After applying, a xenon arc illuminator or endoilluminator is used to polymerize the formulation (if photopolymerizable) and form a thin coat covering the posterior capsule. Saline is then injected into the anterior chamber and the capsular bag is filled with viscoelastic prior to insertion of an intraocular lens is the customary fashion. About one to two years later, after the acute inflammatory response has subsided, the polymer biodegrades.

### EXAMPLE 13

### Mechanical Barriers

As a mechanical barrier, the methods of the present invention create a unique and novel temporary polymer barrier which can alleviate ocular symptoms, while protecting the integrity and normal function of the ocular surface.

Mechanical Barriers, Dry Eye

In dry eye, the ocular surface is not adequately wetted by an optimized tear film. This produces ocular irritation and increases the chance of corneal infection. To prevent the discomfort associated with dry eye and the attendant risk of intraocular infection, PEG based polymer formulations (chemical polymerizing) are applied to the ocular surface in a drop formulation. These biodegradable formulations bioreode in 1-5 days, and are periodically reapplied.

Barriers: Symptomatic Keratoconjuctivitis Sicca

A patient with symptomatic keratoconjuctivitis sicca applies one drop of a low viscosity chemical polymerizing PEG based formulation to the eye using a conventional dropper bottle with perforated end. The drop polymerizes within less than one second following ocular contact, forming a thin barrier layer that maintains clear optical quality of the corneal surface. During about a 1-2 day period, the polymer slowly biodegrades and a drop is then reapplied. Patients have reduced symptoms of foreign body sensation and ocular irritation.

### EXAMPLE 14

### Peg-Based Polymers to Increase Intraocular Pressure

Experiments have confirmed the biocompatibility of the PEG-based polymers of the present invention in a rabbit model and the ability of these polymers to increase the intraocular pressure in rabbits. To establish biocompatibility, the polymers were injected into the anterior chamber angle of the rabbit in the manner described above. After one week, the rabbits were sacrificed, and histopathological examinations performed on the treated eyes. No evidence of intraocular inflammation or corneal endothelial cell loss was noted.

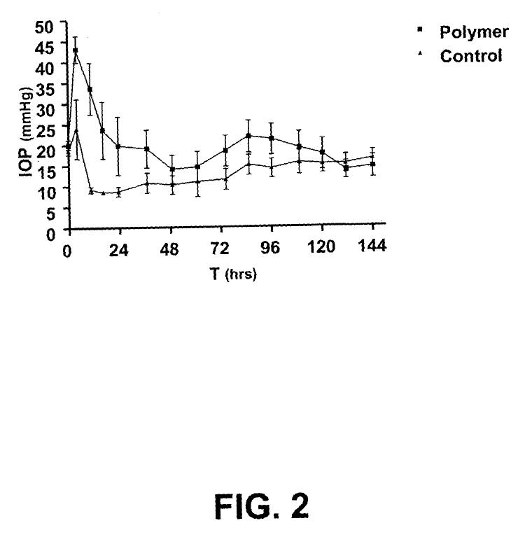

An experiment was then performed comparing the effects on intraocular pressure of a PEG-based polymer with a balanced-salt solution control in eight rabbit eyes. Eight New Zealand White rabbits are given general anesthesia with an intramuscular injection of xylazine and ketamine. Each rabbit has 150 microliters of aqueous removed from one eye with a 30 gauge needle. The treated eyes were then injected with about 50 to 100 microliters of air through a 30 gauge needle to form an air bubble in the anterior chamber. Four eyes were injected with enough PEG-based polymer (approximately 50 to 100 microliters) into the angle to flow over approximately 270° of the angle. Four eyes were injected with 50 to 100 microliters of buffered saline solution. The angles of all eight eyes were irradiated with the fiberoptic endo-illuminator of a Storz Vitrector for 2 to 5 minutes. Finally, the air bubbles were removed with a 30 gauge needle. Four hours after the injection, mean intraocular pressure in the treated eyes was approximately 20 mmHg greater than in control eyes. Some increase in pressure compared to controls was observed up to five days after the procedure (see FIG. 2).

### EXAMPLE 15

### Mammalian Treatment of Insufficient Intraocular Pressure

The methods of the present invention are utilized for treatment of a mammalian eye having deficient intraocular pressure. Anesthesia is administered to the mammal, followed by removal of aqueous from the eye by a needle. An air bubble is injected into the angle through the same paracentesis site as the needle through which the aqueous was removed. A polymer, such as a polymer precursor, is injected into the anterior chamber of the eye toward the angle around either portions of (≦360 degrees), or the entire anterior chamber angle (360 degrees) via one of more paracentesis sites. In embodiments wherein the polymer is photopolymerizable, visible light is applied to the polymer. The air bubble is then removed, such as by aspiration with a needle. Aqueous outflow is retarded, preferably completely, and intraocular pressure is thereby increased. In a specific embodiment, the procedure is repeated.

### References

The following references, to the extent that they provide exemplary procedural or other details supplementary to those set forth herein, are specifically incorporated herein by reference.

### Patents

U.S. Pat. No. 4,328,803

U.S. Pat. No. 4,604,087

U.S. Pat. No. 5,360,399

U.S. Pat. No. 5,614,587

U.S. Pat. No. 5,626,863

U.S. Pat. No. 5,700,794

U.S. Pat. No. 5,801,033

U.S. Pat. No. 5,820,882

U.S. Pat. No. 6,149,931

U.S. Pat. No. 6,274,614

U.S. Pat. No. 6,636,585

### Publications

Alleyene C H Jr, Cawley C M, Barrow D L, Poff B C, Powell M D, Sawhney A S, Dillehay D L, J Neurosurg 88:308-313,1998.

Cadera W, Willis N R. Sodium hyaluronate for postoperativee aphakic choroidal detachment. Can J Ophthalmol 1982; 17:274-275.

Daniele S, Schepens C L. Can chronic bulbar hypotony be responsible for uveal effusion? Report of two cases. Ophthalmic Surg 1989;20:872-875.

Kirchhof B. Retinectomy lowers intraocular pressure in otherwise intractable glaucoma: preliminary results. Ophthalmic Surg 1994;25:262-267.

Koerner F, Niesel P, Konig H. Inflow and outflow relations after intraocular fluid-Healon exchange in a case of chronic ocular hypotension. Int Ophthalmol 1985;8:261-266.

Kuchle M, Naumann G O H. Direct cyclopexy for traumatic cyclodialysis with persisting hypotony. Ophthalmology 1995;102:322-333.

Laurent U B G, Fraser J R E. Turnover of hyaluronate in the aqueous humor and vitreous body of the rabbit. Exp Eye Res 1983;36:493-504.

Lewis H, Aaberg T M, Abrams G W. Causes of failure after initial vitreoretinal surgery for severe proliferative vitreoretinopathy. Am J Ophthalmol 1991;111:8-14.

Lewis H, Aaberg T M. Causes of failure after repeat vitreoretinal surgery for recurrent proliferative vitreoretinopathy. Am J Ophthalmol 1991;111:15-19.

Lewis H; Verdaguer J I. Surgical treatment for chronic hypotony and anterior proliferative vitreoretinopathy. Am J Ophthalmol 1996;122:228-235.

Schubert H D, Denlinger J L, Balazs E A. Exogenous Na-hyaluronate in the anterior chamber of the owl monkey and its effect on the intraocular pressure. Exp Eye Res 1984;39:137-152.

Zarbin M A, Michels R G, Green W R. Dissection of epiciliary tissue to treat chronic hypotony after surgery for retinal detachment with proliferative vitreoretinopathy. Retina 1991;11:208-213.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method for providing a polymer to an ocular defect in a mammal, wherein the ocular defect is other than a retinal break, comprising:
applying over and/or around the ocular defect a non-toxic polymer formulation comprising at least one polymer precursor that is a poly(ethylene glycol) (PEG) based polymer precursor; and
transforming the polymer formulation into a gel-like coat.

2. The method of claim 1, wherein the polymer provides a seal to the ocular defect.

3. The method of claim 1, wherein the ocular defect is at least one opening, incision, wound, hole, tear, gap, notch, aperture, cavity, cut, slit, scratch, injury, lesion, gash, abrasion, break, puncture, perforation, rip, or split in at least one eye tissue.

4. The method of claim 1, wherein the ocular defect is an indirect or direct result of a disease, medical condition, or surgery.

5. The method of claim 3, wherein the surgery is filtration surgery, vitreoretinal surgery, corneal surgery, or scleral buckling surgery.

6. A method of sealing an opening in an eye of a mammal, wherein the opening is not a retinal break, comprising:
applying over and/or around an opening in the eye a non-toxic polymer formulation comprising at least one polymer precursor that is a poly(ethylene glycol) (PEG) based polymer precursor; and
transforming the polymer formulation into a gel-like coat, wherein the coats forms a seal.

7. The method of claim 6, wherein the seal reduces liquid flow from the defect.

8. The method of claim 7, wherein the liquid is aqueous.

9. The method of claim 6, wherein the seal is resistant to intraocular pressure from the eye.

10. The method of claim 6, wherein the opening is optionally sutured.

11. The method of claim 6, wherein the opening in the eye is associated with filtration surgery.

12. The method of claim 6, wherein the opening in the eye is associated with a postoperative glaucoma filtration bleb or conjunctival buttonhole.

13. A method for sealing an opening in an eye of a mammal caused by filtration surgery, comprising:
applying over and/or around an opening in the eye a non-toxic polymer formulation comprising at least one polymer precursor that is a poly(ethylene glycol) (PEG) based polymer precursor; and
transforming the polymer formulation into a gel-like coat, wherein the coat forms a seal.

14. The method of claim 13, wherein the seal reduces liquid flow from the opening.

15. The method of claim 13, wherein the seal is resistant to intraocular pressure from the eye.

16. The method of claim 13, wherein the opening is optionally sutured.

17. The method of claim 13, wherein the opening is a conjunctival incision.

18. A method of sealing a conjunctival incision in the eye of a mammal following filtration surgery, comprising:
applying over and/or around the conjunctival incision a non-toxic polymer formulation comprising at least one polymer precursor that is a poly(ethylene glycol) (PEG) based polymer precursor; and
transforming the polymer formulation into a gel-like coat, wherein the coats forms a seal.

19. The method of claim 18, wherein the seal reduces liquid flow from the defect.

20. The method of claim 18, wherein the seal is resistant to intraocular pressure from the eye.

21. The method of claim 18, wherein the incision is optionally sutured.

22. The method of claim 18, wherein the seal biodegrades after about 2-16 weeks.

23. A method of sealing a leaking corneal wound in the eye of a mammal, comprising:
applying to the wound a non-toxic polymer formulation comprising at least one polymer precursor that is a poly(ethylene glycol) (PEG) based polymer precursor; and
transforming the polymer formulation into a gel-like coat, wherein the coat forms a seal.

24. The method of claim 23, wherein the seal reduces liquid flow from the defect.

25. The method of claim 23, wherein the seal is resistant to intraocular pressure from the eye.

26. The method of claim 23, wherein the wound is optionally sutured.

27. The method of claim 23, wherein the seal biodegrades after about 2-16 weeks.

28. A method of sealing sclerotomies from vitreoretinal surgery in the eye of a mammal, comprising:
applying to a sclerotomic wound a non-toxic polymer formulation comprising at least one polymer precursor that is a poly(ethylene glycol) (PEG) based polymer precursor; and
transforming the polymer formulation into a gel-like coat, wherein the coat forms a seal.

29. The method of claim 28, wherein the seal reduces liquid flow from the defect.

30. The method of claim 28, wherein the seal is resistant to intraocular pressure from the eye.

31. The method of claim 28, wherein the wound is optionally sutured.

32. The method of claim 28, wherein the seal biodegrades after about 2-16 weeks.

33. A method of forming at least one barrier to adhesion and/or of preventing cellular adhesion and proliferation in the eye of a mammal, comprising:
applying to a surface in the eye a non-toxic polymer formulation comprising at least one polymer precursor that is a poly(ethylene glycol)(PEG) based polymer precursor; and
transforming the polymer formulation into a gel-like coat.

34. The method of claim 33, wherein the surface is a filtration site and bleb following filtration surgery.

35. The method of claim 33, wherein the prevention of adhesion reduces scarring of the filtration site and bleb.

36. A method of preventing at least one adhesion from forming between two apposing tissue surfaces in the eye of a mammal, comprising:
applying to apposing surfaces in the eye a non-toxic polymer formulation comprising at least one polymer precursor that is a poly(ethylene glycol)(PEG) based polymer precursor; and
transforming the polymer formulation into a gel-like coat.

37. The method for claim 36, wherein the surface is the filtration site and bleb following filtration surgery.

38. The method of claim 36, wherein the prevention of adhesion reduces scarring of the filtration site and bleb.

39. The method of claim 36, wherein the coat biodegrades after about 2-16 weeks.

40. The method of claim 36, wherein both the outside and inside surfaces of a scleral flap as well as the scleral margins surrounding an excised trabecular segment are coated with the non-toxic polymer formulation.

41. A method for reducing scarring of the filtration site and bleb following filtration surgery in the eye of a mammal, comprising:
preventing and/or reducing at least one post-operative adhesion by applying to apposing surfaces following surgery a non-toxic polymer formulation comprising at least one polymer precursor that is a poly(ethylene glycol)(PEG) based polymer precursor; and
transforming the polymer formulation into a gel-like coat.

42. The method of claim 41, wherein the coat biodegrades after about 2-16 weeks.

43. The method of claim 41, wherein both outside and inside surfaces of a scleral flap and/or the scleral margins surrounding an excised trabecular segment are coated with the non-toxic polymer formulation.

44. A method for preventing at least one adhesion (symblepharons) from forming between the palpebral and bulbar conjunctival surfaces in conjunctival cicatricial disease such as Stevens Johnson Syndrome, comprising:
applying to the surfaces a non-toxic polymer formulation comprising at least one polymer precursor that is a poly(ethylene glycol)(PEG) based polymer precursor: and
transforming the polymer formulation into a gel-like coat.

45. The method of claim 44, wherein the coat biodegrades after about 2-16 weeks.

46. A method for preventing at least one adhesion between the extraocular muscles and at least one adjacent surface in strabismus or scleral buckling surgery, comprising:
applying to exposed extraocular muscles and adjacent tissue or prosthetic surfaces a non-toxic polymer formulation comprising at least one polymer precursor that is a poly(ethylene glycol) (PEG) based polymer precursor; and
transforming the polymer formulation into a gel-like coat.

47. The method of claim 46, wherein the adjacent surface is tenon's capsule, sclera, scleral buckle, or a combination thereof.

48. The method of claim 46, wherein the coat biodegrades after about 2-16 weeks.

49. The method of claim 46, wherein said applying step is repeated before, during, after, or a combination thereof at least some biodegradation of the applied polymer.

50. A method for preventing graft rejection following corneal transplant surgery, comprising:
applying to the endothelial surface of the donor cornea prior to suturing to the host during keratoplasty surgery a non-toxic polymer formulation comprising at least one polymer precursor that is a poly(ethylene glycol)(PEG) based polymer precursor; and
transforming the polymer formulation into a gel-like coat.

51. The method of claim 50, wherein white blood cells are prevented from adhering to the endothelial surface.

52. The method of claim 51, wherein rejection of the corneal transplant is reduced due to the reduced adherence of cellular and/or macromolecular elements.

53. A method of preventing, after cataract surgery, lens epithelial cells from migrating over the posterior lens capsule and causing posterior capsule opacification, comprising:
applying to the posterior capsule prior to and/or just after implantation of the intraocular lens a non-toxic polymer formulation comprising at least one polymer precursor that is a poly(ethylene glycol)(PEG) bsed polymer precursor; and
transforming the polymer formulation into a gel-like coat.

54. The method of claim 53, wherein the coat biodegrades after about 6-48 months.

55. A method of forming a biodegradable barrier in the eye of mammal, comprising:
applying to an ocular surface to be protected a non-toxic polymer formulation comprising at least one polymer precursor that is a poly(ethylene glycol)(PEG) based polymer precursor; and
transforming the polymer formulation into a gel-like coat.

56. The method of claim 55, wherein the barrier is a mechanical barrier.

57. The method of claim 55, wherein the coating of the ocular surface alleviates at least one ocular symptom of irritability and/or protects the integrity and normal function of the ocular surface.

58. The method of claim 55, wherein the coating provides protection against infection.

59. The method of claim 55, wherein the coat alleviates at least one symptom of dry eyes.

60. The method of claim 55, wherein the transforming is by photopolymerization of the polymer precursor.

61. The method of claim 55, wherein the polymer formulation comprises a polymer precursor of the formula:
Pₘ-DₙWₒ-Dₚ-P_{q}
wherein W is a water-soluble polymer; D is a degradable moiety; P is a photopolymerization moiety; m and q are integers from 1 to about 10; o is an integer from 1 to about 100; and n and p are integers from 0 to about 120

62. The method of claim 55, wherein the PEG comprises reactive termini.

63. The method of claim 62, wherein the reactive termini are free radical polymerizable termini.

64. The method of claim 62, wherein the reactive termini are acrylate termini.

65. The method of claim 62, wherein the PEG comprises a long chain PEG having a molecular weight of at least about 8,000 g/mol.

66. The method of claim 62, wherein the PEG comprises a long chain PEG having a molecular weight of at least about 20,000 g/mol.

67. The method of claim 62, wherein the PEG based polymer precursor further comprises degradable regions.

68. The method of claim 67, wherein the degradable regions comprise from about 0.5% to about 20% oligolactic acid.

69. The method of claim 66, wherein the PEG based polymer precursor further comprises about 1% oligolactic acid.

70. The method of claim 55, further comprising applying at least one photoinitiator to the surface.

71. The method of claim 70, wherein the photoinitiator is an eosin Y photoinitiator.

72. The method of claim 70, wherein the formulation further comprises at least one co-catalyst.

73. The method of claim 68, wherein the formulation further comprises at least one photoinitiator and at least one co-catalyst.

74. The method of claim 68, wherein the formulation further comprises at least one photoinitiator, N-vinlypyrrolidone and triethanolamine.

75. The method of claim 55, wherein the transformation is by auto-polymerization of the polymer precursor.

76. The method of claim 55, wherein the polymer formulation comprises a first polymer precursor and a second polymer precursor, the first and second polymer precursors being mutually reactive.

77. The method of claim 76, wherein the first polymer precursor is an amine.

78. The method of claim 77, wherein the amine is a tetra-amino poly(ethylene gylcol)(PEG).

79. The method of claim 76, wherein the first polymer precursor is a protein and the second polymer precursor is a terminally-functionalized poly(ethylene glycol)(PEG).

80. The method of claim 79, wherein the protein is albumin, collagen, or gelatin.

81. The method of claim 80, wherein the protein is albumin.

82. The method of claim 78, wherein the second PEG molecule is a di-N-hydroxysuccinimidyl PEG.

83. The method of claim 76, wherein the second polymer precursor is a hydroxysuccinimidly activated succinate-terminated PEG.

84. The method of claim 76, wherein the second polymer precursor is a hydroxysuccinimidyl activated carbonate-terminated PEG.

85. The method of claim 55, wherein the gel-like coat comprises a biodegradable polymer.

86. A method for increasing intraocular pressure in an eye, comprising the step of limiting the loss of aqueous from said eye.

87. The method of claim 86, wherein said loss from said eye is limited to substantially zero.

88. The method of claim 86, wherein said limiting step is further characterized as applying a biocompatible polymer to said eye.

89. The method of claim 88, wherein the application of said polymer is to the angle of said eye, to the posterior chamber of the eye, or both.

90. The method of claim 88, wherein the application of said polymer obstructs the trabecular meshwork of said eye.

91. The method of claim 88, wherein said polymer is a photopolymerizable polymer.

92. The method of claim 88, wherein said polymer is an autopolymerizable polymer.

93. The method of claim 88, wherein said polymer is a polyethylene glycol-based polymer.

94. The method of claim 93, wherein said polyethylene glycol-based polymer comprises poly(ethylene glycol)-cotrimethylene carbonate-co-lactide (M, 20,000) with acrylated end groups.

95. The method of claim 93, wherein the half-life of said polymer is at least about three days.

96. The method of claim 86, wherein said applying step is further defined as:
removing aqueous from the eye;
administering the polymer precursor; and
polymerizing said polymer.

97. The method of claim 96, wherein the method further comprises the step of applying an apparatus to facilitate directing the polymer precursor into the angle.

98. The method of claim 96, wherein the aqueous is removed from the anterior chamber of said eye.

99. The method of claim 96, wherein said polymerizing step is further defined as applying light to a photopolymerizable polymer.

100. The method of claim 86, wherein said method is repeated following reduction in intraocular pressure in the eye.

101. The method of claim 88, wherein said method is repeated following degradation of the polymer.

102. A method for increasing intraocular pressure in an eye of a mammal, comprising the steps of:
removing aqueous from the eye;
injecting an air bubble into the angle of the eye;
administering a precursor of poly(ethylene glycol)-cotrimethylene carbonate-co-lactide with acrylated end groups into the angle; and
polymerizing the precursor.

103. A method of hindering the loss of aqueous from the eye of an individual comprising administering a biocompatible polymer into the eye.
